(19) United States
(12) Patent Application Publication
(10) Pub. No.: US 2025/0017950 A1
(43) Pub. Date: Jan. 16, 2025

(54) COMPOUNDS FOR TREATING MITOCHONDRIAL DNA DISORDERS

(71) Applicants: UCL BUSINESS LTD, London (GB); UNITED KINGDOM RESEARCH AND INNOVATION, Wiltshire (GB)

(72) Inventors: Antonella Spinazzola, London (GB); Ian Holt, Donostia-San Sebastian (ES); Boris Pantic, London (GB); Daniel Ives, London (GB)

(73) Assignees: UCL BUSINESS LTD, London (GB); UNITED KINGDOM RESEARCH AND INNOVATION, Wiltshire (GB)

(21) Appl. No.: 18/710,471

(22) PCT Filed: Nov. 16, 2022
(86) PCT No.: PCT/GB2022/052910

§ 371 Date: May 15, 2024

(30) Foreign Application Priority Data
Nov. 16, 2021 (GB) 2116499.1

(51) Int. Cl. A61K 31/7004; A61K 31/19; A61K 31/70; A61K 38/50

(52) U.S. Cl. CPC A61K 31/7004; A61K 31/19; A61K 31/70; A61K 38/50; C12Y 305/01001

(57) ABSTRACT

There is described a compound for use in the treatment of a mitochondrial DNA disorder, wherein the compound is a glycolysis inhibitor, an inhibitor of glutamine consumption or is L-asparaginase or pegaspargase. The glycolysis inhibitor may be a glucose analogue such as 2-deoxy-D-glucose (2DG) or 5-thioglucose (5TG). Also described is a method of treating a mitochondrial DNA disorder comprising administering a therapeutically effective amount of the compound above to a patient suffering from a mitochondrial DNA disorder. In addition, there is described a method of reducing the mtDNA heteroplasmy in the cells of a patient comprising administering a therapeutically effective amount of the compound above to the patient.

### FIELD OF THE INVENTION

The present invention relates to compounds and compositions for use in the treatment of mitochondrial DNA (mtDNA) disorders, and in particular those disorders where individuals carry a level of mutant mtDNA sufficient to impair mitochondrial function and cause disease.

### BACKGROUND TO THE INVENTION

Most of the energy the body needs is converted in the parts of the cell called mitochondria from ingested food. Critical for this process is the DNA present in the mitochondria (mtDNA). Hence, defects in the mtDNA cause an energy crisis, with consequent disease. Most cells contain thousands of copies of the mtDNA. Often, when a mutation occurs, it affects some, but not all the copies of mtDNA, a state known as heteroplasmy. Crucially, most heteroplasmic deleterious mtDNA variants are recessive; that is, the mutants produce biochemical and clinical phenotypes only at relatively high levels (usually in excess of 50% of all the mtDNAs). This means that it is not necessary to eradicate all the mutant mtDNA to restore mitochondrial function; instead, a modest decrease in mutant load should be sufficient to transition from a disease to a healthy state.

At present, there is no effective treatment, still less a cure, for mtDNA disorders. Drugs in development aim to increase mitochondrial mass rather than correct the underlying problem of the mutant mtDNA, and therefore will only ever limit disease progression, and not reverse it.

WO 2015/157409 relates to targeting platinum-containing therapeutic agents to mitochondria to treat cancer. However, this document does not disclose the use of inhibitors of glycolysis or glutamine metabolism as a therapeutic agent to treat heteroplasmic mtDNA disorders. Nor does it explain how decreasing glycolysis would be appropriate when mitochondrial dysfunction increases reliance on glycolysis; i.e., the patent makes no claim that 2-deoxy-D-glucose (“2DG”) is appropriate for treating mtDNA disorders.

Therefore, there is a need for a treatment for mtDNA disorders that reverses the disease and can be rapidly translated to the clinic.

### SUMMARY OF THE INVENTION

In a first aspect of the invention, there is provided a compound for use in the treatment of a mitochondrial DNA disorder, wherein the compound is a glycolysis inhibitor.

In a related aspect, there is provided the use of a compound in the manufacture of a medicament for treating a mitochondrial DNA disorder, wherein the compound is a glycolysis inhibitor.

The inventors have surprisingly found that glycolysis inhibitors (such as 2-deoxy-D-glucose (2DG), 5-thioglucose (5TG) and oxamate) select wild-type mtDNA molecules in multiple cell types and restore the mitochondrial respiratory capacity. The positive selection of wild-type mtDNA molecules involves the inhibition of replication of the mutant mtDNA but not the wild-type mtDNA. Mechanistically, the selection of the wild-type mtDNAs depends on restriction of glucose and/or glutamine, as this forces the mitochondria to be dependent on their own energy producing capacity and anabolic resources, thus disadvantaging the mutant mtDNA.

By restricting glucose utilisation, the compound supports the replication of functional mtDNAs, as glucose-fuelled respiration is critical for mtDNA replication in control cells, when glucose and glutamine are scarce or cannot be utilized. The outcome in these circumstances is that non-mutant mtDNAs can sustain replication and thus propagate, whereas the replication of the mutant molecules is impaired. This results in a decreased level of mutant mtDNA, restored replication and improved mitochondrial function.

Glucose is metabolised through glycolysis. The glycolysis pathway and its metabolic interconnection with lactate, the pentose phosphate pathway, extracellular glucose uptake, and the TCA cycle in mitochondria is shown in FIG. 18. The glycolysis inhibitor can be any compound that causes inhibition of one or more parts of the glycolysis pathway. For example, the glycolysis inhibitor may be an inhibitor of one of the enzymes in the glycolysis pathway. Alternatively, the glycolysis inhibitor may be a compound that disrupts the normal functioning of the glycolysis pathway and limits glycolytic flux. Compounds of this type include but are not limited to inhibitors of glucose uptake and analogues of the metabolites that form from the glycolysis pathway. A specific example of the latter type is oxamate that inhibits lactate dehydrogenase, an enzyme directly adjacent to, but not part of, glycolysis. It has been shown that oxamate has the same effect on mutant mtDNA load and the mitochondrial respiratory chain protein levels as glucose analogues (FIG. 17).

In some embodiments, the glycolysis inhibitor is an inhibitor of an enzyme selected from a glucose transporter, hexokinase, glucose-6-phosphate dehydrogenase, transketolase, phosphoglucose isomerase, phosphofructokinase, aldolase, triosephosphate isomerase, glyceraldehyde-3-phosphate dehydrogenase, phosphoglycerate kinase, phosphoglycerate mutase, enolase, pyruvate kinase, lactate dehydrogenase or a lactate transporter. Inhibitors of these enzymes are well known to those skilled in the art. In various embodiments, the glycolysis inhibitor is an inhibitor of an enzyme selected from glucose transporter 1, hexokinase, glucose-6-phosphate dehydrogenase, aldolase, phosphofructokinase, glyceraldehyde-3-phosphate dehydrogenase, phosphoglycerate kinase, pyruvate kinase or lactate dehydrogenase.

In some embodiments, the glycolysis inhibitor is an inhibitor of a glucose transporter, such as glucose transporter 1 (GLUT1). Such inhibitors include but are not limited to Phloretin, Quercetin, Fasentin, STF31 (Chan D A et al., Science Translational Medicine, 3:94ra70 (2011)) and WZB117 (Liu Y et al., Molecular Cancer Therapeutics, 11:1672-1682 (2012)).

In some embodiments, the glycolysis inhibitor is an inhibitor of hexokinase. Such inhibitors include but are not limited to 3-bromopyruvic acid, 3-bromopyruvate, D-Mannoheptulose, N-acetylglucosamine, Imatinib, Lonidamine, SID 856002 (Ebselen), SID 17387000, SID 24785302, SID 3716597, SID 24830882, SID 16952891, SID 22401406, SID 24797131, SID 17386310 and SID 14728414 (Sharlow E R et al., PLOS Neglected Tropical Diseases, 4(4): e659).

In some embodiments, the glycolysis inhibitor is an inhibitor of glucose-6-phosphate dehydrogenase. Such inhibitors include but are not limited to 6-aminonicotinamide (6AN) and dehydroepiandrosterone (DHEA).

In some embodiments, the glycolysis inhibitor is an inhibitor of transketolase. Such inhibitors include but are not limited to oxythiamine chloride hydrochloride, p-hydroxyphenylpyruvate and diphenylurea derivatives T2, T2A, T2B, T2C, T2D and T2E (Obiol-Pardo C et al., PLOS ONE 7(3): e32276 (2012)).

In some embodiments, the glycolysis inhibitor is an inhibitor of phosphoglucose isomerase. Such inhibitors include but are not limited to d-arabinose-5-phosphate derivatives, ST090269, ST082230, ST078079, 5251606, 7993994, 6877084, ST060239, 7963836,6125285,5150036,7950244,9064882,5116964,5224468,9074873,9193149, 5331342, 7745039, ST093058 and ST057360 (Mota S G R et al., SLAS DISCOVERY: Advancing the Science of Drug Discovery. 2018; 23(10):1051-1059.)

In some embodiments, the glycolysis inhibitor is an inhibitor of phosphofructokinase. Such inhibitors include but are not limited to 3-(3-pyridinyl)-1-(4-pyridinyl)-2-propen-1-one (3PO) and PFK158 (Granchi C et al., Bioorganic and Medicinal Chemistry Letters, 24:4915-4925 (2014)), Auranofin, ZINC04887558 (N4A, 5, 6, 7, 8-tetrahydroxy-2-(4-hydroxyphenyl) chromen-4-one), YN1 (7, 8-dihydroxy-3-(4-hydroxyphenyl) chromen-4-one) and YZ9 (ethyl 7-hydroxy-2-oxochromene-3-carboxylate).

In some embodiments, the glycolysis inhibitor is an inhibitor of aldolase. Such inhibitors include but are not limited to bisphosphonate inhibitors, such as naphthalene 2,6-bisphosphate, as well as 2,6-dihydroxy-1-naphthaldehyde, 2-phosphate-naphthalene 6-bisphosphonate, 2-naphthol 6-bisphosphonate, and 1-phosphate-benzene 4-bisphosphonate.

In some embodiments, the glycolysis inhibitor is an inhibitor of triosephosphate isomerase. Such inhibitors include but are not limited to phosphoglycolohydroxamic acid and the two compounds below identified by Marsh et al. (Marsh L. et al., International Journal of Medicinal Chemistry, vol. 2014, Article ID 469125, 6 pages, 2014):

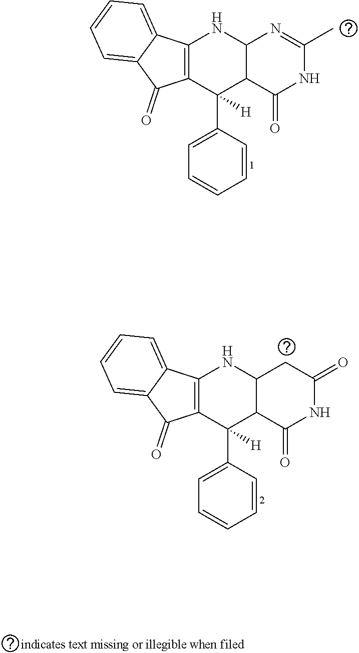 [SMILES: CC1=NC2NC3=C(C(=O)c4ccccc43)[C@H](c3ccccc3)C2C(=O)N1 ; O=C1CC2NC3=C(C(=O)c4ccccc43)[C@H](c3ccccc3)C2C(=O)N1]

In some embodiments, the glycolysis inhibitor is an inhibitor of glyceraldehyde-3-phosphate dehydrogenase. Such inhibitors include but are not limited to koningic acid (aka heptelidic acid), arsenate and iodoacetate, 3BrPA, DC-5163, Saframycin A and methylglyoxal.

In some embodiments, the glycolysis inhibitor is an inhibitor of phosphoglycerate kinase. Such inhibitors include but are not limited to NG52 (Wen-Liang Wang, et al. Acta Pharmacol Sin. 2021 April; 42(4):633-640) and salicylates.

In some embodiments, the glycolysis inhibitor is an inhibitor of phosphoglycerate mutase. Such inhibitors include but are not limited to MJE3 (Li N et al., Onco Targets Ther. 2020; 13:1787-1795), PGMI-004A (CAS No.: 1313738-90-7), N-Xanthone Benzenesulfonamides, anthraquinone and HKB99 (Liang Q et al., Acta Pharmacol Sin. 2021 January; 42(1):115-119).

In some embodiments, the glycolysis inhibitor is an inhibitor of enolase. Such inhibitors include but are not limited to fluoride, SF2312 (Leonard P G et al., Nat Chem Biol. 2016 December: 12(12):1053-1058), mefloauine, and phosphonoacetohydroxamate.

In some embodiments, the glycolysis inhibitor is an inhibitor of pyruvate kinase. Such inhibitors include but are not limited to Shikonin, alkannin, and the PKM peptide inhibitors TLN-232 and CAP-232.

In some embodiments, the glycolysis inhibitor is an inhibitor of lactate dehydrogenase. Such inhibitors include but are not limited to oxamic acid, oxamate and NHI-1 (Granchi C et al., Journal of Medicinal Chemistry, 54:1599-1612 (2011)), FX11 (CAS No. 213971-34-7), Quinoline 3-sulfonamides, and monoclonal antibodies Trastuzumab, Cetuximab that target LDH. In one embodiment, the glycolysis inhibitor is oxamic acid or oxamate.

In some embodiments, the glycolysis inhibitor is an inhibitor of lactate transport (export from the cell, via transporters such as MCT1 and MCT4). Such inhibitors include but are not limited to Bevacizumab, salicylate and its derivatives, including 3-phenylpropionate (3PP) and 3-(2-methylphenyl)-propionate (2M3PP) (Bosshart, P. D. et al., Commun Chem 4, 128 (2021)).

In certain embodiments, the glycolysis inhibitor is a glucose analogue. Such analogues are well known to those skilled in the art. Preferably, the glucose analogue is a D-glucose analogue. The glucose analogue may be a glucose molecule that had been modified so that it cannot undergo further glycolysis. The glucose analogue may act to competitively inhibit the production of glucose-6-phosphate from glucose. Suitable glucose analogues include but are not limited to 2-deoxy-D-glucose (2DG), 2-fluoro-2-deoxy-d-glucose (2-FG), 2-chloro-2-deoxy-d-glucose (2-CG), 2-bromo-2-deoxy-d-glucose (2-BG), 5-thioglucose (5TG), 2-fluoro-d-mannose (2-FM), acetyl 2-DG analogues, 1,5 anhydro-D-fructose, the glucose analog 6-0 benzyl-D-galactose, C3361 (Blume, M. et al., 2011 The FASEB Journal, 25:1218-1229) and WP1122 (3,6-di-O-acetyl-2-deoxy-d-glucose-Priebe W et al., Neuro-Oncology. 2018; 20:vi86). In certain embodiments, the glucose analogue is 2-deoxy-D-glucose (2DG) or 5-thioglucose (5TG).

In some embodiments, the compound is an inhibitor of glutamine consumption/utilization. 2-DG reduces glutamine consumption (as well as inhibiting glycolysis) (Wang, F. et al., 2018 Cell Metabolism 28, 463-475 e464), and removing glutamine from the growth medium decreases the mutant mtDNA load for m.3243A>G, albeit not as efficiently as 2-DG (Pantic, B. et al., 2021 Nat Commun. 12(1):6997). As a result, in one aspect of the invention, there is provided a compound for use in the treatment of a mitochondrial DNA disorder, wherein the compound is an inhibitor of glutamine consumption. Further, there is provided the use of a compound in the manufacture of a medicament for treating a mitochondrial DNA disorder, wherein the compound is an inhibitor of glutamine consumption.

The compound is used to treat a mitochondrial DNA disorder. Mitochondrial DNA disorders are disorders caused by mutations in either the mitochondrial DNA (mtDNA) or nuclear DNA (nDNA) that lead to dysfunction of the mitochondria and inadequate production of energy in the form of ATP. The mitochondrial DNA encodes 13 hydrophobic proteins that are essential subunits of oxidative phosphorylation complexes (I, III, IV & V), along with 22 tRNAs and the 2 rRNAs essential for their translation. Mutations of mitochondrial DNA include point mutations and deletions.

mtDNA disorders can present at any age and features include, but are not restricted to, ptosis, exercise intolerance, myopathy, pigmentary retinopathy, cardiomyopathy, sensorineural deafness, diabetes mellitus, parkinsonism. In many cases, the phenotypes fall in specific clinical syndromes such as: Maternally Inherited Diabetes and Deafness (MIDD), Mitochondrial Myopathy (MM), Chronic Progressive External Ophthalmoplegia (CPEO), Maternal Inherited Leigh Syndrome (MILS), Mitochondrial Encephalomyopathy Lactic Acidosis and Stroke-Like Episodes (MELAS), Pearson Syndrome (PS), Kearns-Sayre Syndrome (KSS), Myoclonic Epilepsy with Ragged-Red Fibers (MERRF), Neurogenic weakness with Ataxia and Retinitis Pigmentosa (NARP), Mitochondrial NeuroGastroIntestinal Encephalopathy-like (MNGIE-like), Sensory Neural Hearing Loss (SNHL), Sudden Infant Death Syndrome (SIDS), Focal Segmental Glomerulosclerosis (FSGS).

The multi-copy nature of the mitochondrial genome leads to complicated genetics. mtDNA mutations can be either heteroplasmic (where both mutated and wild type mtDNA co-exist within the cell) or homoplasmic (only mutated species are present). In the heteroplasmic state, for a mutated species to cause a phenotypic effect, the proportion of mutated mtDNA and wild-type mtDNA (heteroplasmy) needs to reach a level where wild-type mtDNA can no longer compensate for the biochemical deficit of the mitochondria with mutant mtDNA. The threshold of mutated mtDNA required to cause a detectable phenotype depends on the mutation type. Broadly speaking, however, single large-scale mtDNA deletions that remove several genes generally require lower heteroplasmy (˜60%) of mutated mtDNA to produce severe respiratory deficiency than point mutations such as m.3243A>G mt-tRNALeu(UUR) which decreases the rate of protein synthesis, usually requiring heteroplasmy greater than 80%.

Since first being described 30 years ago, hundreds of mtDNA mutations have been shown to be associated with human disease. Ninety-seven point mutants have been confirmed as pathogenic according to one database, (https://www.mitomap.org/foswiki/bin/view/MITOMAP/ConfirmedMutations) and most of these are heteroplasmic. They include individual point mutations in transfer and messenger RNA genes, and protein encoding genes that cause a wide range of overlapping diseases. Among the most common point mutations are m.3243A>G, m.8344A>G, m.8993T>G and m.8993T>C. Known mutations include m.583G>A, m.616T>C, m.1494C>T, m.1555A>G, m.1606G>A, m.1630A>G, m.1644G>A, m.3243A>G, m.3243A>T, m.3256C>T, m.3258T>C, m.3260A>G, m.3271T>C, m.3273delT, m.3280A>G, m.3291T>C, m.3302A>G, m.3303C>T, m.3376G>A, m.3460G>A, m.3635G>A, m.3697G>A, m.3700G>A, m.3733G>A, m.3890G>A, m.3902_3908 ACCTTGCinv, m.4171C>A, m.4298G>A, m.4300A>G, m.4308G>A, m.4332G>A, m.4450G>A, m.5521G>A, m.5537_5538insT, m.5650G>A, m.5690A>G, m.5703G>A, m.5728T>C, m.7445A>G, m.7445A>G, m.7471_7472insC, m.7497G>A, m.7510T>C, m.7511T>C, m.8306T>C, m.8313G>A, m.8340G>A, m.8344A>G, m.8356T>C, m.8363G>A, m.8528T>C, m.8851T>C, m.8969G>A, m.8993T>C, m.8993T>G, m.9035T>C, m.9155A>G, m.9176T>C, m.9176T>G, m.9185T>C, m.9205_9206delTA, m.10010T>C, m.10158T>C, m.10191T>C, m.10197G>A, m.10663T>C, m.11777C>A, m.11778G>A, m.12147G>A, m.12201T>C, m.12258C>A, m.12276G>A, m.12294G>A, m.12315G>A, m.12316G>A, m.12706T>C, m.13042G>A, m.13051G>A, m.13094T>C, m.13379A>C, m.13513G>A, m.13514A>G, m.14459G>A, m.14482C>A, m.14482C>G, m.14484T>C, m.14487T>C, m.14495A>G, m.14568C>T, m.14674T>C, m.14709T>C, m.14710G>A, m.14849T>C, m.15579A>G and m.15990C>T.

Additionally, deletions of various sizes in the mtDNA are invariably heteroplasmic. They result in the loss of all or part of mitochondrial transfer, messenger and ribosomal genes. The most frequent example in patients is a ˜5 kb deletion spanning ATPase 8 to ND5—the so called “common deletion” nt.8467_13446del4977.

Additionally, duplications of various sizes in the mtDNA are invariably heteroplasmic. They result in gene fusions and imbalances in the products of mtDNA and can disrupt mtDNA maintenance and expression or cell metabolism.

Additionally, mutations in cis regulatory elements, such as those found in the major non-coding region of mtDNA that can disrupt mtDNA maintenance and expression or cell metabolism.

Therefore, the mtDNA disorder can also be associated with: a deletion that encompasses all or part of a mitochondrial transfer RNA gene; a deletion that encompasses all or part of a mitochondrial ribosomal RNA gene; a deletion that encompasses all or part of one of the 13 mitochondrial protein encoding genes; a point mutation, a deletion or other rearrangement that affects a regulatory “cis-element” in the mtDNA, such as those found in the major non-coding or ‘control’ region; or a rearrangement of the mtDNA that disrupts its maintenance or expression.

People with mitochondrial DNA disorders can present at any age with almost any affected body system; however, the brain, muscles, heart, liver, nerves, eyes, ears and kidneys are the organs and tissues most commonly affected. In children, the most common phenotypes include poor growth, developmental delay, learning disabilities, autism, while neuromuscular involvement and problems with vision and/or hearing are typical of the adult-onset form. In some cases, movement disorders, such as dystonia, Parkinson disease, parkinsonism, chorea, as well as dementia, ataxia or multi-organ failure are manifested. Stroke like episodes, cardiomyopathy and enchepahomyopathy are frequent causes of premature death.

The mitochondrial DNA disorder treated by the compound is a heteroplasmic mitochondrial DNA disorder.

In various embodiments, the mitochondrial DNA disorder is associated with a point mutation, such as m.3243A>G, m.8344A>G, m.8993T>G and m.8993T>C. In particular embodiments, the mitochondrial DNA disorder is associated with the mitochondrial DNA m.3243A>G mutation.

In a number of embodiments, the mitochondrial DNA disorder is associated with a deletion of mtDNA, such as nt.8467_13446del4977.

As indicated above, the use of the compound causes positive selection of wild-type molecules as it selectively inhibits the replication of mutant mtDNA. This has the effect of reducing the heteroplasmy so that there is a lower proportion of mutated mtDNA to wild-type mtDNA. This reduced intracellular heteroplasmy helps to alleviate the mitochondrial DNA disorder.

In the present invention, the use of the compound in the treatment of a mitochondrial DNA disorder does not cover the treatment of cancer. In the treatment of a mitochondrial DNA disorder, the compound causes a reduction in the heteroplasmy in cells. It does not kill cells that contain mutant mitochondrial DNA as is the goal of cancer treatment. The inventors clearly show that the compounds induce intra-cellular selection of functional mitochondria and mtDNAs. Instead, cancer treatments aim to favour non-cancerous cells over cancerous cells, i.e. through inter-cellular competition.

In a related aspect, there is provided a compound for use in the treatment of a mitochondrial DNA disorder, wherein the compound is L-asparaginase or pegaspargase. Further, there is provided the use of a compound in the manufacture of a medicament for treating a mitochondrial DNA disorder, wherein the compound is L-asparaginase or pegaspargase.

The inventors have also shown that cells containing a relatively high load of mutant mtDNA have increased glutamine utilisation and that glutamine restriction is important for the selection of wild-type mtDNA. One of the fates of glutamine in cells is asparagine: this becomes indispensable in glutamine-restricted conditions, either because of low supply or increased utilization. The inventors have shown that 2DG inhibits asparagine synthetase. One of the effects of low asparagine is to depress mitochondrial DNA replication. Therefore, decreasing asparagine availability by treating the mutant cells with L-asparaginase or pegaspargase is thought to favour the wild-type mtDNA over mutant mtDNA.

The description above relating to the treatment of a mitochondrial DNA disorder is equally applicable to this aspect in which the compound is L-asparaginase or pegaspargase.

The compounds of the present invention can be used, alone or in combination with other therapeutic agents, in the treatment of various conditions or disease states. The present invention includes the use of a combination of a compound of the invention and one or more additional therapeutic agent(s). In some embodiments, the one or more additional therapeutic agents are selected from the group consisting of mannose, asparginase, oxamate, pegaspargase, and metformin. In some cases, 2DG or 5TG can be combined with compound(s) that modulate glucose metabolism, such as the antidiabetic metformin, to produce a synergistic effect (Horakova O., et al. 2019 Sci Rep. 9(1) 6156; Zhao J, et al. 2019 Cell Death Discov 5:76). Exemplary combinations can include, but are not limited to, 2DG+mannose, 2DG+asparginase, 2DG+oxamate, 2DG+pegaspargase, 2DG+metformin, 2DG+mannose+oxamate, 5TG+mannose, 5TG+asparginase, 5TG+oxamate, 5TG+pegaspargase, 5TG+metformin, and 5TG+mannose+oxamate. The compounds and additional therapeutic agents may be administered simultaneously, concurrently or sequentially. Additionally, simultaneous administration may be carried out by mixing the compounds prior to administration or by administering the compounds at the same point in time but at different anatomic sites or using different routes of administration. The phrases “concurrent administration,” “co-administration,” “simultaneous administration,” and “administered simultaneously” mean that the compounds are administered in combination.

Advantageously, the inventors have shown that mannose depresses the ER stress without interfering in the selection of wild-type mtDNAs, providing a more tolerable treatment option. As a result, in some embodiments, the compound is for administration in combination with mannose. In particular embodiments, the compound is 2DG for administration in combination with mannose (2DG+mannose). In particular embodiments, the compound is 5TG for administration in combination with mannose (5TG+mannose). The compound(s) may be administered simultaneously (either in the same dosage form or in separate dosage forms) or sequentially. The compounds may be administered simultaneously, concurrently or sequentially. Additionally, simultaneous administration may be carried out by mixing the compounds prior to administration or by administering the compounds at the same point in time but at different anatomic sites or using different routes of administration.

The compound may be formulated into a pharmaceutical composition comprising the compound and one or more pharmaceutically acceptable excipients. The present invention also includes pharmaceutical compositions comprising an amount of: (a) a compound of the invention or a pharmaceutically acceptable salt thereof; (b) a second therapeutic agent; and (c) one or more pharmaceutically acceptable excipients.

Acceptable excipients for therapeutic use are well known in the pharmaceutical art, and are described, for example, in Remington's Pharmaceutical Sciences, Mack Publishing Co. (A. R. Gennaro edit. 1985). The choice of pharmaceutical excipient can be selected with regard to the intended route of administration and standard pharmaceutical practice.

The pharmaceutically acceptable excipient encompasses any suitable dosage form that is acceptable for administration to a patient. The excipient can be a solid, a liquid, or both, and may be formulated with the compound as a unit-dose composition, for example, a tablet, which can contain from 0.05% to 95% by weight of the active compounds. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension. A compound of the invention may be coupled with suitable polymers as targetable drug carriers. Other pharmacologically active substances can also be present.

The pharmaceutical compositions may comprise as, or in addition to, the excipient, any suitable binder, lubricant, suspending agent, diluent, coating agent or solubilising agent.

Preservatives, stabilizers and dyes may be provided in the pharmaceutical composition. Examples of preservatives include sodium benzoate, sorbic acid and esters of p-hydroxybenzoic acid. Antioxidants and suspending agents may be also used.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically. In particular embodiments, the composition is for oral administration.

Oral administration of a solid dose form may be, for example, presented in discrete units, such as hard or soft capsules, pills, cachets, lozenges, or tablets, each containing a predetermined amount of at least one compound of the present invention.

In another embodiment, the oral administration may be in a powder or granule form. In another embodiment, the oral dose form is sub-lingual, such as, for example, a lozenge. In such solid dosage forms, the compounds of the invention are ordinarily combined with one or more excipients. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise excipients, such as wetting, emulsifying, suspending, flavoring (e.g., sweetening), and/or perfuming agents.

For young patients or those patients with difficulty swallowing, compositions may be provided in granule, powder or liquid form.

In another embodiment, the present invention comprises a parenteral dose form. “Parenteral administration” includes, for example, subcutaneous injections, intravenous injections, intraperitoneal injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J. Pharm. Sci., 88 (10), 955-958, by Finnin and Morgan (October 1999).

Formulations suitable for topical administration to the eye include, for example, eye drops wherein the compound of this invention is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (e.g., absorbable gel sponges, collagen) and non-biodegradable (e.g., silicone) implants, wafers, lenses and particulate or vesicular systems, such as niosomes or liposomes. A polymer such as cross-linked polyacrylic acid, polyvinyl alcohol, hyaluronic acid, a cellulosic polymer, for example, hydroxypropylmethyl cellulose, hydroxyethyl cellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

The amount or dose of the pharmaceutical composition that is administered should be sufficient to alleviate the disease in vivo. The dose will be determined by the efficacy of the particular formulation, as well as the body weight of the subject to be treated. In some embodiments, the amount or dose of the pharmaceutical composition that is administered is calculated/adjusted based on monitoring of the patient following administration of an earlier dose of the pharmaceutical composition,

The dose of the pharmaceutical composition will also be determined by the existence, nature, and extent of any toxicity and/or adverse side effects that might accompany the administration of a particular formulation. Typically, a physician will decide the dosage of the composition with which to treat each individual subject, taking into consideration a variety of factors, such as age, body weight, general health, medical condition, diet, sex, compound/formulation to be administered, activity of the particular compound employed, route of administration, and the severity of the condition being treated. The appropriate dosage can be determined by one skilled in the art. By way of non-limiting example, the total dose of the active ingredient in the composition of the present invention (e.g., 2DG or 5TG) can be from about 1 mg/kg to about 500 mg/kg body weight of the subject being treated, administered in single or divided doses. In one embodiment, the total daily dose of the compound(s) of the invention is typically from about 1 mg/kg to about 100 mg/kg body weight of the subject being treated per day. In one embodiment, the total daily dose of the compound(s) of the invention is typically from about 2 mg/kg to about 100 mg/kg body weight of the subject being treated per day. In another embodiment, total daily doses of the compounds of the invention will range from 5 to 50 mg/kg body weight, and in another embodiment it will be from 10 to 30 mg/kg. In one embodiment, dosing is from 1 to 10 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

In alternative embodiments, the total dose of the active ingredient in the composition of the present invention (e.g., 2DG or 5TG) can be from about 2 to about 500 mg/kg body weight of the subject being treated, from about 5 to about 300 mg/kg, from about 10 mg/kg to about 200 mg/kg, and from about 20 mg to about 100 mg/kg body weight. Furthermore, the total dose of the active ingredient in the composition of the present invention (e.g., 2DG or 5TG) can be from about 5 to about 180 mg/kg body weight of the subject being treated, from about 10 mg/kg to about 120 mg/kg, and from about 20 mg to about 60 mg/kg body weight.

In some embodiments, the total dose of asparaginase (e.g., L-asparaginase) in the composition is from about 200 to about 1000 international units/m2.

For the treatment of the conditions referred to above, the compound(s) of the invention can be administered as compound per se. Alternatively, pharmaceutically acceptable salts are suitable for medical applications because of their greater aqueous solubility relative to the parent compound.

In a preferred embodiment, the composition of the present invention is administered daily or intermittently (e.g., once or twice per week, every other day, every other week, etc.), although it is expected that both the dose and frequency will be reduced once the wild-type mtDNA has reached a level that restores mitochondrial function. Employing an intermittent dosing strategy may reduce side effects and/or toxicity associated with the administration of agents, and could prove fully effective as intermittent dosing was used to decrease the load of mutant mtDNA in cells (Pantic B., et al., 2021 Nat Commun. 12(1):6997). Thus, in some embodiments, the intermittent dosing strategy comprises administering an initial dose of the compound(s) of the invention to the subject and administering subsequent doses of the compound(s) of the invention approximately every other day. In some embodiments, the intermittent dosing strategy comprises administering an initial dose of the compound(s) of the invention to the subject and administering subsequent doses of the compound(s) of the invention approximately twice a week. In some embodiments, the intermittent dosing strategy comprises administering an initial dose of the compound(s) of the invention to the subject and administering subsequent doses of the compound(s) of the invention approximately once a week. In some embodiments, the intermittent dosing strategy comprises administering an initial dose of the compound(s) of the invention to the subject and administering subsequent doses of the compound(s) of the invention approximately once every two weeks. In some embodiments, the intermittent dosing strategy comprises administering an initial dose of the compound(s) of the invention to the subject and administering subsequent doses of the compound(s) of the invention approximately two days every two weeks. In some embodiments, the intermittent dosing strategy comprises administering an initial dose of the compound(s) of the invention to the subject and administering subsequent doses of the compound(s) of the invention approximately three days every two weeks. In some embodiments, the intermittent dosing strategy comprises administering an initial dose of the compound(s) of the invention to the subject and administering subsequent doses of the compound(s) of the invention approximately once every three weeks. In some embodiments, the intermittent dosing strategy comprises administering an initial dose of the compound(s) of the invention to the subject and administering subsequent doses of the compound(s) of the invention approximately once every four weeks. In some embodiments, the compound(s) of the invention are administered using one dosing strategy (as described above) and an additional therapeutic agent is administered using a different dosing strategy (as described above). For example, in some embodiments, the compound(s) of the invention are administered using an intermittent dosing strategy (e.g., every other day) and the additional therapeutic agent is administered weekly.

For oral administration, the compositions may be provided in the form of tablets containing from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

In some embodiments, a combination of two or more of the compounds described above are used in the treatment of a mitochondrial DNA disorder. In some embodiments, a combination of two or more of the compounds described above and one or more additional therapeutic agents are used in the treatment of a mitochondrial DNA disorder.

In some embodiments, one or a combination of two or more of the compounds described above are used in the treatment of accumulations of mutant/defective mtDNA. In some embodiments, a combination of two or more of the compounds described above and one or more additional therapeutic agents are used in the treatment of accumulations of mutant/defective mtDNA.

There is also provided a method of treating a mitochondrial DNA disorder comprising administering a therapeutically effective amount of the compound described herein to a patient suffering from a mitochondrial DNA disorder. Suitable patients according to the present invention include mammalian patients. Mammals according to the present invention include, but are not limited to, canine, feline, bovine, caprine, equine, ovine, porcine, rodents, lagomorphs, primates, and the like, and encompass mammals in utero. In one embodiment, humans are suitable patients. Human patients may be of either gender and at any stage of development.

In some embodiments, the method further comprises administering a therapeutically effective amount of mannose to the patient. The amount or dose of mannose may be as described above for the compounds.

When the mtDNA disorder is “treated” in the above method, this means that one or more symptoms of the mtDNA disorder are ameliorated. It does not mean that the symptoms of the mtDNA disorder are completely remedied so that they are no longer present in the patient, although in some methods, this may be the case. The method of treating results in one or more of the symptoms of the mtDNA disorder being less severe than before treatment.

A “therapeutically effective amount” refers to an amount effective, at dosages and for periods of time necessary, to achieve the desired therapeutic result, such as reducing the heteroplasmy in cells (so as to lead to a level sufficient to ameliorate the pathologies associated with the mitochondrial DNA disorder).

In a related aspect, there is provided a method of reducing the mtDNA heteroplasmy in the cells of a patient comprising administering a therapeutically effective amount of the compound described herein to the patient.

In a further aspect, there is provided a method of reducing the mtDNA heteroplasmy in a cell comprising administering a therapeutically effective amount of the compound described herein to the cell.

### BRIEF DESCRIPTION OF THE DRAWINGS

The invention will now be described in detail by way of example only with reference to the figures in which:

FIG. 1 shows that 2-deoxy-D-glucose (2DG) and 5-thioglucose (5TG) induce a shift from m.3243A>G to wild-type mtDNA on three nuclear backgrounds and restore mitochondrial respiratory capacity. The level of mutant (m.3243A>G) mtDNA was determined by pyrosequencing or restriction fragment length polymorphism analysis of DNA isolated from cells subjected to intermittent treatment with vehicle (black lines) or 10 mM 2DG (solid green line) or 10 mM, 5TG (red line) (panels a-f). A549 adenocarcinoma cells heteroplasmic for m.3243A/G (a-c). b, After 42 days, A549 cells subject to continuous 10 mM 2DG treatment (solid green line) were grown for a further 42 days in medium lacking 2DG (broken green line). d, Rhabdomyosarcoma (Myo.RD) m.3243A>G. e, f, Primary human fibroblasts of patients (P1 and P2) carrying m.3243A>G. Number of independent experiments: n=5 for a; n=6 for c; n=7 and 5 for 5TG and 2DG, respectively, panel d; n=12 and 9 for 2DG and 5TG, respectively, panel e; n=8 and 6 for 2DG and 5TG, respectively, panel f; panels b and j, n=1. Data represent the mean value±SD; two-sided Mann-Whitney test; ns=not significant; a, ns P(A549 Veh vs 2DG 4w)=0.0952; a, **P(A549 Veh vs 2DG 8w)=0.0079; c, **P(A549 Veh vs. 5TG 4w)=0.0079; c, **P(A549 Veh vs 5TG 8w)=0.0022; d, **P(Myo.RD Veh vs. 5TG 2w)=0.00079; d, ***P(Myo.RD Veh vs 5TG 4w)=0.0006; d, **P(Myo.RD Veh vs. 2DG 4w)=0.0079; e, *P(P1 Veh vs. 5TG 2w)=0.0286; ***P(P1 Veh vs 5TG 4w)=0.0006; **P(P1 Veh vs 5TG 8w)=0.0079; *P(P1 Veh vs 2DG 2w)=0.0286; ***P(P1 Veh vs 2DG 4w)=0.000041; ***P(P1 Veh vs. 2DG 8w)=0.000011; f, **P(P2 Veh vs 2DG 2w)=0.0022; f, **P(P2 Veh vs. 2DG 4w)=0.0022; f, **P(P2 Veh vs. 5TG 2w)=0.0022; **P(P2 Veh vs. 5TG 4w)=0.0079. Panels a, c, d, e and f with all the data points are showed in FIG. 17. Increased wild-type mtDNA was accompanied by elevated mitochondrial translation in P1 vs. control (C1) (g); and increased OXPHOS proteins (h) and respiration (i) in P1 and P2. g, Newly synthesized mitochondrial-encoded proteins in P1 fibroblasts were detected by 35S-methionine pulse-labelling, n=3 independent experiments; putative mitochondrial polypeptides labels are indicated to the side of the gel (NADH dehydrogenase subunits, ND1-6/L; cytochrome c oxidase subunits COX1-3; ATP synthase subunits 6 and 8; and cytochrome b); and coomassie staining of the gel shows equal protein loading. h, steady-state levels of selected oxidative phosphorylation (OXPHOS) proteins (COX2 and NDUFB8) were detected by immunobloting, Vinculin (VCL) was used as loading control; n=11 independent experiments. i, Oxygen (O2) consumption was measured by flux-analysis (Seahorse Instrumentation); data in arbitrary units (AU) represent the mean value±SD of n=4 and 3 independent experiments for P1 and P2 respectively; two-sided unpaired t-test with Welch's correction: **P (P1 Veh vs 2DG basal OCR)=0.0014, **P(P1 Veh vs 2DG maximal OCR)=0.0055, **P(P2 Veh vs 2DG basal OCR)=0.0014, **P(P2 Veh vs 2DG maximal OCR)=0.0057. g-i, Fibroblasts of P1 and P2 were treated intermittently with 2DG for 8 and 4 weeks, respectively. j, Non-dividing P1 and P2 fibroblasts (by contact inhibition) were treated intermittently with the small molecules for 6 and 4 weeks, respectively. Immunoblots of P2 cellular protein for OXPHOS subunits are shown beside the chart showing the change in mutant load with time.

FIG. 2 shows minimal and escalating 2DG doses decreasing the m.3243A>G load, and overall shift rate. a, Fibroblasts were subjected to intermittent treatment with vehicle or different concentrations of 2DG (0.1-10 mM) with 5 mM glucose, or (b) an escalating dose regime, in which the first 2 rounds of treatment were 0.1 mM 2DG, followed by 2 rounds of 0.25 mM 2DG, and subsequent rounds of 0.5 mM 2DG. c, Calculated heteroplasmy shift rates in A549 and Myo.RD cells, and primary fibroblasts, treated with and without glucose analogues (2DG or 5TG) or 1 mM glucose, no glutamine, or oligomycin. Shift rate describes the rate of change of heteroplasmy over time, accounting for the fact that it is measured as a percentage and hence follows sigmoidal dynamics. Number of independent experiments for 2DG and 5TG treatments: A549, n=12; MyoRD, n=12; fibroblasts, n=38; for oligomycin A549, n=4. Data represent the mean±SEM. P-values are given from a two-sided One-sample median test for A549 and Myo-RD, and a two-sided Wilcoxon signed-rank test for fibroblasts against the null hypothesis that glucose analogues have no effect on mutant load. P-values: A549=8.30E-07; Myo-RD: 3.90E-0.5; Fibroblasts: 7.276E-12; A549+Oligo: 0.001.

FIG. 3 shows that 2DG inhibits mtDNA replication and autophagy in fibroblasts with high mutant loads. a, Control (grey) and patient, P1 and P2, (green) fibroblasts were treated with vehicle (veh) or 10 mM 2DG for 48 hours, for the final 6 hours some cells were treated additionally with 50 μM choloroquine (CLQ) to block autophagy, and cellular proteins were analysed by immunobloting. The chart indicates the ratio of the non-lipidated (I) and lipidated (II) autophagosome marker LC3, normalized to that of vehicle and CLQ treated cells after normalization to the loading control. Data represent the mean value±SD of n=5 and 4 independent experiments for P1 and P2, respectively. Two-sided unpaired t-test with Welch's correction: ns P(Controls CLQ vs 2DG+CLQ)=0.6600; ***P(P1 CLQ vs. 2DG+CLQ)=0.000003; *P(P2 CLQ vs. 2DG+CLQ)=0.0122. b, Control (C1-C3) and patient (P1 and P2) fibroblasts treated with and without 10 mM 2DG or 5TG for 48 h and labelled with 50 μM 5-bromo-2′-deoxyuridine (BrdU) for 13-16 h. After fixing, the cells were stained green with anti-BrdU and red with anti-DNA antibodies. Bar=10 m. Cells with little or no cytoplasmic BrdU positive foci were scored as ‘mtDNA replication inhibited’ and ≥500 cells were counted from 6 independent experiments for 5TG, and >10 for 2DG (see also FIG. 10); data represent the mean value±SD. One-way ANOVA: ***P(Controls vs. P1, 2DG; Controls vs. P2, 2DG; Controls vs. P1, 5TG; Controls vs. P2, 5TG; P1 Veh vs. 2DG; P1 Veh vs 5TG; P2 Veh vs 2DG; P2 Veh vs. 5TG)<0.000001; ns P(Controls Veh vs. 2DG; Controls Veh vs. 5TG)>0.9999. c, m.3243A>G mutants treated with and without 5TG were labelled with BrdU and imaged as panel b for P2 fibroblasts or analysed for LC3 as panel a for P1 (n=6 independent experiments, 3 each for P1 and P2). Bar=10 μm. d, BrdU labelling as panel b, indicating the recovery of mtDNA synthesis in mutant (P1 and P2) cells after treatment with 2DG for 4 weeks compared to a 48 h treatment (n=5 independent experiments, 3 for P2 and 2 for P1); data are expressed as BrdU foci (in the cytoplasm, i.e. newly synthesized mtDNA) per cell. Images are for P2 cells treated with 2DG for 48 h or intermittently for 4 weeks (see also FIG. 9e for P1 and P2). Bar=15 μm. e, Patient (P2) fibroblasts were treated with 10 mM 2DG or vehicle. After DNA immunoprecipitation with an anti-BrdU antibody, a region of mtDNA encompassing bp 3243 was amplified and subjected to restriction fragment length polymorphism analysis. Pie charts represent the % of m.3243A>G (MUT—light green) and wild-type (WT—dark green) mtDNA in captured DNA fractions. Data represent the mean value±SEM of n=4 independent experiments. Two-sided unpaired t-test with Welch's correction: *P(Wild-type IP Veh vs 2DG)=0.0018. To the right, a representative agarose gel showing analysis of the anti-BrdU antibody precipitated material after PCR amplification and ApaI digestion, together with a mock-precipitation without antibody (No Ab), and the source DNAs prior to immunoprecipitation (Input). Without 2DG, the ratio of BrdU-containing wild-type mtDNA (IP fraction) to the total (Input) was 1.03:1 (n=3 independent experiments). In contrast, with 2DG the ratio was 3.25:1 (n=3 experiments); see FIG. 10f for individual data points and chart. Hence, mtDNA synthesis (BrdU incorporation) is equal for mutant and wild-type molecules without treatment, whereas synthesis occurs predominantly on wild-type mtDNAs in the presence of 2DG.

FIG. 4 shows bioenergetics underlies the effects of 2DG and 5TG on mtDNA replication and autophagy in fibroblasts with high mutant load, and inhibition of OXPHOS reverses the direction of segregation. a, ATP levels in Control (grey), P1 (orange) and P2 (light orange) fibroblasts treated without and with 10 mM 2DG for 48 h and 1 μM oligomycin (Oligo) for 90 minutes, or both compounds. Data represent the mean value±SD of n=4 and 3 independent experiments for P1 and P2, respectively. One-way ANOVA: ***P(Control Veh vs. 2DG; P1 Veh vs. 2DG; P2 Veh vs. 2DG)<0.000001; ***P(Control vs. P1, 2DG; Control vs. P2, 2DG)<0.000001; ***P(Control 2DG vs. 2DG+Oligo)<0.000001; ns P(P1 2DG vs. 2DG+Oligo)=0.5867; ns P(P2 2DG vs. 2DG+Oligo)=0.1885. b, Proportion of depolarized cells in Control, P1 and P2 fibroblasts treated without and with 2DG, 5TG or OLI, normalized to 5 μM FCCP-treated cells (100% depolarized cell). Data represent the mean value±SD of n=7 and 6 independent experiments for P1 and P2 cells treated with 2DG, respectively; and n=3 and 3 for 5TG. One-way ANOVA: ns P(Control Veh vs. Oligo)=0.0942; ns P(Control Veh vs. 2DG)=0.2868; ns P(Control Veh vs. 5TG)=0.886; ***P(P1 Veh vs. Oligo)<0.000001; ***P(P1 Veh vs. 2DG)<0.000001; **P(P1 Veh vs. 5TG)=0.0032;***P(P2 Veh vs. Oligo)<0.000001; ***P(P2 Veh vs. 2DG)=0.0009; ns P (P2 Veh vs. 5TG)=0.2631. c, P1 fibroblasts were treated with and without 2DG, 5TG and CLQ as indicated. Extracted cellular protein was immunoblotted for AMP kinase (AMPK), and the activated form phosphorylated at threonine 172 (pAMPK) and vinculin as the loading control; n=4 independent experiments. d, Mitochondrial DNA synthesis in control fibroblasts treated with (mid-grey) and without (light grey) 10 mM 2DG, 1 μM rotenone (black), or 10 mM 2DG and 1 μM rotenone (striped mid-grey) for 48 h, BrdU labelling and analysis as FIG. 3b; Data represent the mean value±SD of n=4 independent experiments. One-way ANOVA: ns P(Controls Veh vs. 2DG)=0.9787; ***P(Controls Veh vs. 2DG+Rot)<0.000001; ns P(Controls Veh vs. Rot)=0.9628; ***P (Controls 2DG vs. 2DG+Rot)<0.000001; ns P(Controls 2DG vs. Rot)=0.8211; ***P(Controls 2DG+Rot vs. Controls Rot)<0.000001. Bar=10 μm. e, Analysis of autophagic flux in control cells treated with and without 10 mM 2DG and 1 μM rotenone (rot) for 48 h, immunoblot and data analysis as FIG. 3a. Controls without (mid-grey) and with (light grey) CLQ. Data represent the mean value±SD of n=3 independent experiments. One-way ANOVA: ns P (Control CLQ Veh vs. CLQ+2DG)=0.1403; ***P (Control CLQ Veh vs. CLQ+2DG+Rot; Control CLQ+2DG+Rot vs. CLQ+Rot)<0.000001; ***P (Control CLQ Veh vs CLQ+Rot)=0.0005. A549 cells were treated with vehicle (black), 10 mM 2DG (blue) or 2DG and 1 μM rotenone (green) (f,g), or oligomycin (purple) alone (h), and m.3243A>G mutant load measured at intervals by pyrosequencing. In panel g the vehicle treated cells were split at day 84 and a new 2DG or 2DG+rotenone treatment commenced (vehicle treated values beyond day 84 are shown in panel f, but not g). h: Data represent the mean value±SD of n=4 independent experiments. Two-sided, Mann-Whitney: *P(A549 Veh vs. Oligomycin 4w; A549 Veh vs. Oligomycin 8w)=0.0286.

FIG. 5 shows that glutamine restriction preferentially inhibits mtDNA replication in m.3243A>G fibroblasts. Control (C1) and patient (P1) fibroblasts were grown in medium containing or lacking glutamine and different concentrations of glucose for 24 h, with 50 μM BrdU to label newly synthesized mtDNA for the final 13 h; in some cases 0.5 μM rotenone was added together with the BrdU to assess the additional impact of inhibition of complex I. Cells were fixed and immunostained to detect DNA that had incorporated BrdU (green) together with TOM20 staining (red) of the mitochondrial network (merge). HG, 25 mM glucose; LG, 1 mM glucose; rot, rotenone; -Gln, no glutamine. Bar=10 μm; n=4 independent experiments.

FIG. 6 shows that glutamine restriction induces a shift from m.3243A>G to wild-type mtDNA in mutant fibroblast, that is more pronounced in low glucose conditions, mimicking the effect of 2DG. a, P2 fibroblasts were treated intermittently with vehicle (black line) or 10 mM 2DG (green line) or 25 mM glucose no glutamine (HG-Gln, light blue line) or 1 mM Glucose no glutamine (LG-Gln, dark blue line). The proportion of mutant mtDNA was plotted against time. Data represent the mean value±SD of n=2 experiments for 2DG and HG-Gln, and n=3 for LG-Gln. For all the conditions one experiment was performed on contact inhibited cells. b, Steady-state levels of the nuclear (NDUFB8) and mitochondrial (COX2) OXPHOS subunits after 4 weeks of intermittent treatment detected by immunoblotting. Proliferating cell nuclear antigen (PCNA) was used as an indicator of cell proliferation, and HSP60 as an indicator of mitochondrial mass, and VCL as the loading control; n=2 independent experiments. c, m.3243A>G mutants (P2) treated with vehicle or LG-Gln for 48 hours or 4 weeks were labelled with BrdU and imaged as previously (FIG. 3b). The BrdU signals indicate the recovery of the mtDNA synthesis after 4 weeks vs 48 hours. n=2 independent experiments; bar=15 μM. In the accompanying chart the data are expressed as cytoplasmic BrdU foci (i.e. replicating mtDNAs) per cell (green columns).

FIG. 7 shows that 2DG promotes segregation to wild-type mtDNA by restricting glutamine (Gln) and glucose (Glc) utilization. Mitochondrial DNA replication can be supported by glucose-fuelled respiration, glycolysis or glutamine (see text for details). The mitochondrial dysfunction caused by m.3243A>G disables the first of these, and consequently increases glycolysis and glutamine consumption (16). 2DG restricts glucose and glutamine metabolism (18). Hence, 2DG forces cells/mitochondria to rely on pyruvate for mitochondrial energy production to sustain mtDNA replication (FIG. 4d). This provides a model that explains how 2DG drives the positive selection of organelles with wild-type mtDNA (FIG. 2c), as mitochondria with m.3243A>G are respiratory (complex I) deficient and so largely unable to utilise pyruvate. Bright green filled arrows/mitochondria-highly active; light green filled arrows/mitochondria—low activity; light red filled arrows/mitochondria—impaired activity.

FIG. 8 shows effects of glucose analogues on mutant load, glycolysis, OXPHOS proteins, and mtDNA copy number. a, A549 cells carrying 76% m.3243A>G were grown, in DMEM supplemented with 0 (red line) or 25 mM (black line) glucose and 10% FBS. DNA was harvested at intervals and the mutant load determined by pyrosequecing (see methods) and plotted against time. b, The effects of 2DG and 5TG on the extracellular acidification rate (ECAR) were measured using a XF flux-analyzer (Seahorse instrumentation) in A549 and Myo.RD by injecting the compounds directly on the plate through one of the ports of the cartridge. Based on the assay, glycolysis was inhibited by 60-80% by the compounds. Data are derived from 3 independent experiments for each cell line and errors are ±SD. One-way ANOVA: ***P (A549 Veh vs 2DG ECAR)=0.000123; ***P (A549 Veh vs 5TG ECAR)=0.000034; ***P(Myo.RD Veh vs 2DG ECAR)=0.00001; ***P (Myo-RD Veh vs 5TG ECAR)=0.000008; **P (A549 Veh vs 2DG OCR)=0.0087; **P (A549 Veh vs 5TG OCR)=0.0049; *P (Myo.RD Veh vs 2DG OCR)=0.0156; **P (Myo.RD Veh vs 5TG OCR)=0.0017. c, P2 fibroblasts were treated intermittently with or without 2DG or 5TG and the level of heteroplasmy was measured by pyrosequencing and the proportion of mutant mtDNA plotted against time. d, The progressive increase in wild-type mtDNA associated with 2DG or 5TG treatment (c) is accompanied by increasing amounts of OXPHOS proteins, based on Western blots analysis of the OXPHOS subunits COX2 (complex IV) and NDUFB8 (complex I) in whole cell lysates at the indicated time-points. Vinculin (VCL) is shown as loading control; the same progressive increase in OXPHOS proteins was observed in two additional independent experiments. e, Heteroplasmy changes were not associated with any significant change in mtDNA copy number in fibroblasts treated with 2DG. Data represent the mean value±SD of n=2 and 3 independent experiments for P1 and P2, respectively. One-way ANOVA: ns P(P1/P2 Veh vs 2DG 48 h)=0.1213; ns P(P1/P2 Veh vs 2DG 4w)=0.8729.

FIG. 9 shows effects of 2DG on cell growth and survival. a, b, 2DG did not cause an increase in cell death, based on visual detection of cellular debris or detached cells, or calcein labelling of viable fibroblasts after 48 h exposure to 2DG, or LDH levels in spent medium harvested from control and patient-derived fibroblasts after treating with or without 2DG for 48 h, using as control cells lysed with Triton X-100; n=3 independent experiments. Bar=300 μM. c, 2DG inhibits the growth of fibroblasts with mutant and wild-type mtDNA, based on proliferation rate determined using an Incucyte™-adapted incubator. Cells were imaged every hour and the proliferation rate was determined by analysing the sequence of images with the manufacturer's software to generate growth curves expressing cell density over time. The start of the 2DG treatment is indicated by the green bar; vehicle—black line and 2DG-treated cells—green line. d, Mutant cells (P2) treated with 2DG or vehicle in proliferating or contact inhibition conditions for 4 weeks. Levels of NDUFB8 are increased in 2DG treated samples without an increase of mitochondrial mass (HSP60). PCNA, a marker of cell proliferation, is markedly downregulated in the 2DG-treated samples; n=3 and 2 independent experiments for 2DG and 5TG, respectively.

FIG. 10 shows that glucose analogues inhibit mtDNA replication and autophagy in fibroblasts carrying high levels of m.3243A>G; and replication is restored after long-term treatment. a, 2DG inhibits autophagy in cells with high mutant load, the inhibitory effect is greater in P1 (92% m.3243A>G) than P2 (85% m.3243A>G) fibroblasts. Cells were treated with or without 10 mM 2DG for 48 h, and with or without CLQ for the final 6 hours. Extracted protein was immunolabelled for the lysosomal marker LC3B, with vinculin (VCL) as the loading control; n=4 independent experiments. b, P2 fibroblasts treated with vehicle or 10 mM 2DG or 48 h immunostained for DNA (green) and the mitochondrial network (TOM20, red); n=3 independent experiments. Bar=10 m. c, Representative images (widefield microscopy) of P1, P2 and C1 fibroblasts treated with vehicle or 2DG and immunostained for BrdU (green) and TOM20 (red); n=6 independent experiments. Bar=20 m. d, P1 fibroblasts treated with vehicle or 10 mM 5TG or 48 h and immunostained for BrdU (green); n=6 independent experiments. Bar=m. e, Representative images (confocal microscopy) showing BrdU labelling in P1 cells treated with vehicle (that does not inhibit mtDNA synthesis) or 2DG for 4 weeks, with the BrdU present for the final 13 h of a 48 h period of exposure to 10 mM 2DG. Bar=10 μm. P2 cells treated with vehicle for 1 week; 2DG for 1 week or 2DG for 4 weeks, as indicated. Bar=15 m. f, Individual data points of the BrdU-DNA immunoprecipitations (IP) described in FIG. 3e. Plots represent the proportion of wild-type mtDNA before (Input) and after (IP) capture by the anti-BrdU antibody from P2 cells, treated with or without 2DG. Data represent the mean value±SEM of n=3 independent experiments. Unpaired, two-sided t-test with Welsh's correction *P(2DG Input vs IP)=0.019.

FIG. 11 shows that the glycolytic inhibitor KA does not inhibit mtDNA synthesis in m.3243A>G fibroblasts. Representative images of Controls (C1 and C2) and Patients (P1 and P2) fibroblasts grown for 24 h in medium with and without 0.5 μM Koningic acid (KA) and where indicated 1 μM rotenone (rot). For the final 13 h 50 μM BrdU was included in the medium after which the cells were fixed and stained with anti-BrdU antibody—green signal and merged with anti-Tom20 (red—mitochondrial network); n=4 independent experiments. Bar=20 m.

FIG. 12 shows that galactose has a modest inhibitory effect on mtDNA replication in m.3243A>G, while it causes extensive cell death within 48 hours. a, In contrast to 2DG and 5TG, substitution of 25 mM glucose (HG) with 5 mM galactose (Gal) does not inhibit mtDNA synthesis based on BrdU labelling in P1 fibroblasts, n=4 independent experiments. Bar=20 m. b, Few cells survived 48 h after replacing glucose with galactose (n=4 independent experiments), and c, LDH indicates there was extensive cell death in m.3243A>G cells (P1 and P2) after 48 h in galactose (values expressed as a proportion of 100% cell lysis with Triton-X 100). N=2 independent experiments for each cell line (C1, C2, P1 and P2). Bar=100 m.

FIG. 13 shows that combined glutamine and glucose restriction mimics the inhibitory effect of 2DG on cell growth in m.3243A>G mutant and control cells. Representative images of Controls (C2) and Patient (P2) fibroblasts grown for 48 h in medium containing, or lacking, glutamine, and different concentrations of glucose for 48 h, or 10 mM 2DG. While glucose restriction slows the growth of mutants but not control cells, additional withdrawal of glutamine markedly inhibits the cell growth in both mutant and controls, as does 2DG. HG, 25 mM glucose; LG, 1 mM glucose; —Gln, no glutamine. N=3 independent experiments. Bar=100 m.

FIG. 14 shows that 2DG induced ER-stress is higher in control cells than those carrying m.3243A>G and is alleviated by mannose supplementation, which does not prevent positive selection of wild-type mtDNA. a, GRP78 was detected by immunofluorescence in control fibroblasts (C1, C2) and fibroblasts carrying m.3243A>G (P1 and P2), treated with and without 10 mM 2DG for 48 h in the presence and absence of mannose (M); n=5 independent experiments. Bar=50 m. b, Control (C1, C2) and mutant fibroblasts (P1, P2) treated with 10 mM 2DG with or without 10 mM mannose (M) for 48 h, or treated with vehicle. 5 μM choloroquine (CLQ) was added for the final 6 hours of the incubation where indicated to block autophagy. Cellular proteins, GRP78, AMPK, pAMPK and the loading control vinculin (VCL) were detected by immunoblotting; n=3 independent experiments. c, P2 and, P1 cells were subjected to 8 pulses of vehicle, 5TG or 2DG with and without mannose, over the course of 4 weeks and the mutant load assayed every 2 weeks. Data represent the mean value±SD of n=3 independent experiments. One way ANOVA: ***P(P1 Veh vs 2DG 4w)=0.000021; ***P(P1 Veh vs 5TG 4w)=0.000009; ***P(P1 Veh vs 2DG+Man 4w)=0.000008; ns P(P1 2DG vs 2DG+Man 4w)=0.540348; **P(P2 Veh vs. 2DG 4w)=0.00104; ***P(P2 Veh vs. 5TG 4w)=0.000144; ***P(P2 Veh vs 2DG+Man 4w)=0.000127; ns P(P2 2DG vs 2DG+Man 4w)=0.198559. Duplicate charts showing individual data points are showed immediately below. Data points to the 2DG & 5TG treatments are also included in main FIGS. 1e and 1f.

FIG. 15 shows intermittent treatment regimes. a, The treatment regime for A549 and RD cybrids comprised weekly cycles of 48 h in the presence of drug or modified medium (first pulse) followed by 24 h without drug or non-restrictive medium (release), 72 h with drug or modified medium (second pulse) and a further 24 h recovery, i.e. two pulses per week. b, m.3243A>G fibroblasts treatment involved two pulses of 48 h separated by 24 or 48 h without drug or modified treatment (release).

FIG. 16 shows individual replicates with means connected for some panels of the FIGS. 1, and 4. Panel a corresponds to FIG. 1a; b to FIG. 1c; c to FIG. 1d; d to FIG. 1e; e to FIG. 1f; f to FIG. 4h.

FIG. 17 shows experiments with oxamate as the inhibitory compound. a) Oxamate inhibits mtDNA replication in cells carrying high levels of mutant mtDNA. b) Oxamate selects wild-type mtDNA in heteroplasmic cells (n=3 independent experiments). c) The decrease in mutant load increases the levels of OXPHOS subunits after four weeks of treatment.

FIG. 18 shows the glycolytic pathway and its metabolic interconnection with the pentose phosphate pathway. The solid arrows indicate glycolytic reactions, whereas the dashed arrows show the pentose phosphate pathway. HK: hexokinase; PGI: phosphoglucose isomerase; PFK: phosphofructokinase; TPI: triosephosphate isomerase; GAPDH: glyceraldehyde-3-phosphate dehydrogenase; PGK: phosphoglycerate kinase; PGM: phosphoglycerate mutase; PK: pyruvate kinase; PDH: pyruvate dehydrogenase; LDH: lactate dehydrogenase.

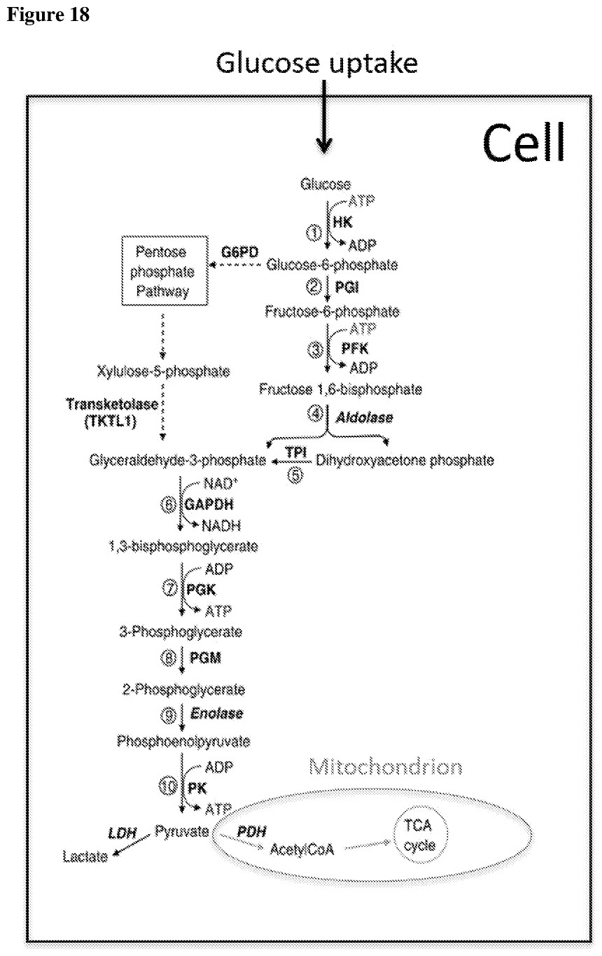

### DETAILED DESCRIPTION OF THE INVENTION

### Examples

### Summary

Pathological variants of human mitochondrial DNA (mtDNA) typically co-exist with wild-type molecules, but the factors driving the selection of each are not understood. Because mitochondrial fitness does not favour the propagation of functional mtDNAs in disease states, the inventors sought to create conditions where it would be advantageous. Glucose and glutamine consumption are increased in mtDNA dysfunction, and so the inventors targeted the use of both in cells carrying the pathogenic m.3243A>G variant with 2-deoxy-D-glucose (2DG), or the related 5-thioglucose. Here, the inventors show that both compounds selected wild-type over mutant mtDNA, restoring mtDNA expression and respiration. Mechanistically, 2DG selectively inhibits the replication of mutant mtDNA; and glutamine is the key target metabolite, as its withdrawal, too, suppresses mtDNA synthesis in mutant cells. Additionally, by restricting glucose utilization, 2DG supports functional mtDNAs, as glucose-fuelled respiration is critical for mtDNA replication in control cells, when glucose and glutamine are scarce. Hence, the inventors demonstrate that mitochondrial fitness dictates metabolite preference for mtDNA replication; consequently, interventions that restrict metabolite availability can suppress pathological mtDNAs, by coupling mitochondrial fitness and replication.

Glucose Analogues Favour Wild-Type mtDNA Molecules in Multiple Cell Types and Restore Mitochondrial Respiratory Function

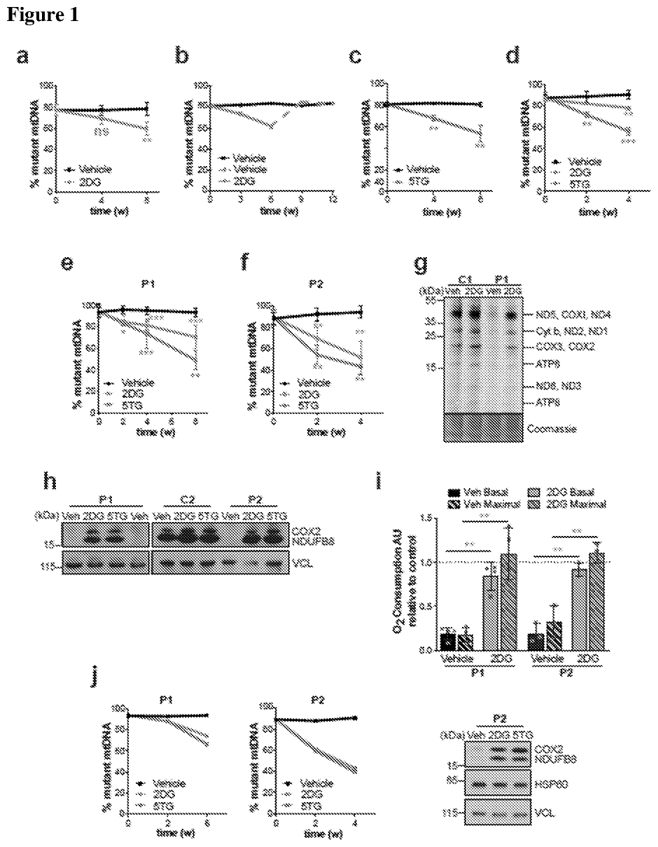

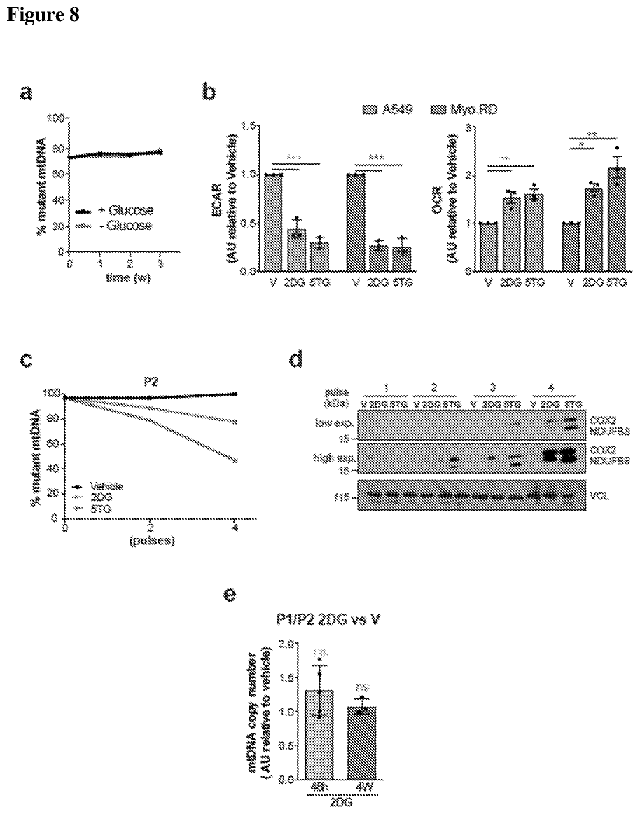

Increased glucose and glutamine utilization supports cells with mutant mtDNA, so the inventors sought to promote active selection of wild-type mtDNA by restricting their usage with 2-deoxy-D-glucose (2DG). In stable, heteroplasmic A549 m.3243A>G cells, 2DG treatments led to a modest, but significant decrease in the proportion of mutated mtDNA (FIG. 1a), whereas glucose restriction did not alter the level of mutant molecules (FIG. 8a). Moreover, when 2DG was withdrawn from the growth medium, m.3243A>G returned to its original level (FIG. 1b). These data suggested that the changes in heteroplasmy level were a consequence of 2DG, rather than the result of random drift to wild-type mtDNA, or selective death of cells with high mutant loads. 5TG, which represses the glycolytic flux to a similar extent to 2DG (FIG. 8b), also decreased the mutant load in A549 cells (FIG. 1c).

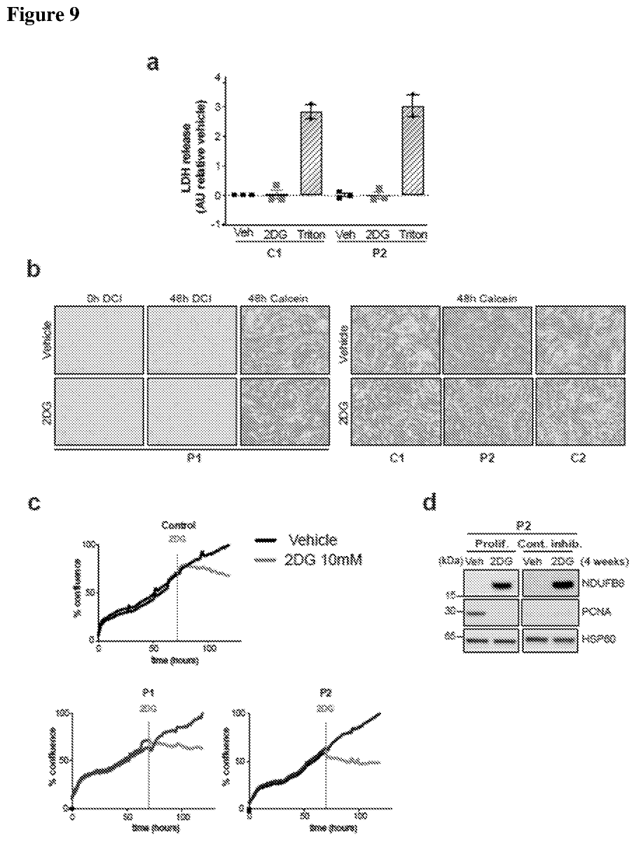

Next, the inventors tested the effect of the compounds on the same mutant mtDNA in another nuclear background (rhabdomyosarcoma, Myo.RD), which has never been reported to select wild-type mtDNA spontaneously. Here too, both chemicals induced segregation to wild-type mtDNA (FIG. 1d); and they were most effective at decreasing m.3243A>G mutant load in primary patient-derived fibroblasts (P1 and P2) (FIG. 1e,f, FIG. 8c). Further analysis of the patient-derived cells indicated that the increase in wild-type mtDNA was sufficient to restore mitochondrial translation, OXPHOS components and respiration to control levels (FIG. 1g-i, and FIG. 8d). The decrease in m.3243A>G was not accompanied by alteration of the mtDNA copy number (FIG. 8e), nor did 2DG treatment cause cell death (FIG. 9a,b). Segregation to wild-type mtDNA was also independent of cellular proliferation, as 2DG slowed cell growth equally in mutant and control cells (FIG. 9c), and 2DG and 5TG decreased the mutant load in fully confluent (contact inhibited) P1 and P2 fibroblasts, again accompanied by restoration of OXPHOS protein levels (FIG. 1j and FIG. 9d). Combined, these data indicated that the glucose analogues induced the selection of wild-type mtDNA in three different cell types (nuclear backgrounds) and on mtDNAs derived from three unrelated affected individuals, via intracellular selection; thereby achieving the goal of overriding the cells' propensity to maintain or select mutant mtDNAs.

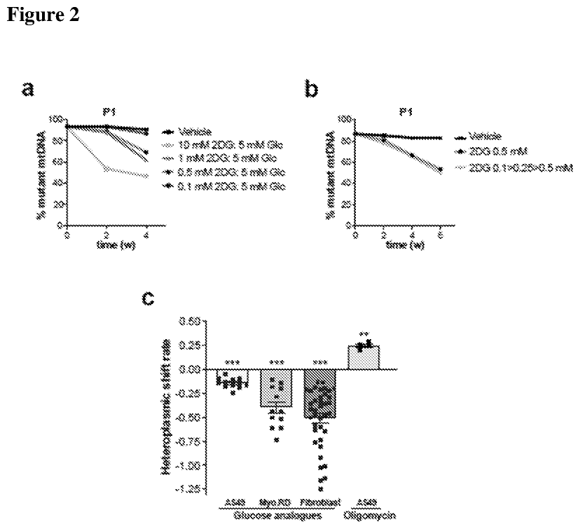

The preceding experiments all employed 10 mM 2DG in medium containing 25 mM glucose and so lower doses of 2DG were tested in combination with 5 mM glucose, a more physiological concentration. A dose of 0.5 mM 2DG proved sufficient to decrease the proportion of m.3243A>G (FIG. 2a), and 2DG was equally effective at lowering mutant loads when an escalating dose regime was applied (FIG. 2b).

The consistent effect of the 2DG and 5TG on the mutant load was clear evidence of them inducing active selection (FIG. 1, 2a,b). To quantify the selective advantage to wild-type mtDNAs conferred by the glucose analogues, the inventors calculated the heteroplasmy shift rate, using the entire time series dataset for each treatment (FIG. 2c). In the patient-derived fibroblasts, the glucose analogues produced a shift of −0.50, which corresponds to a change from 57% to 45% mutant mtDNA in one week. Hence, the inventors' findings demonstrated that active selection produced pronounced and rapid changes in heteroplasmy in human somatic cells.

2DG and 5TG Restrict mtDNA Replication in Cells with High Mutant Load

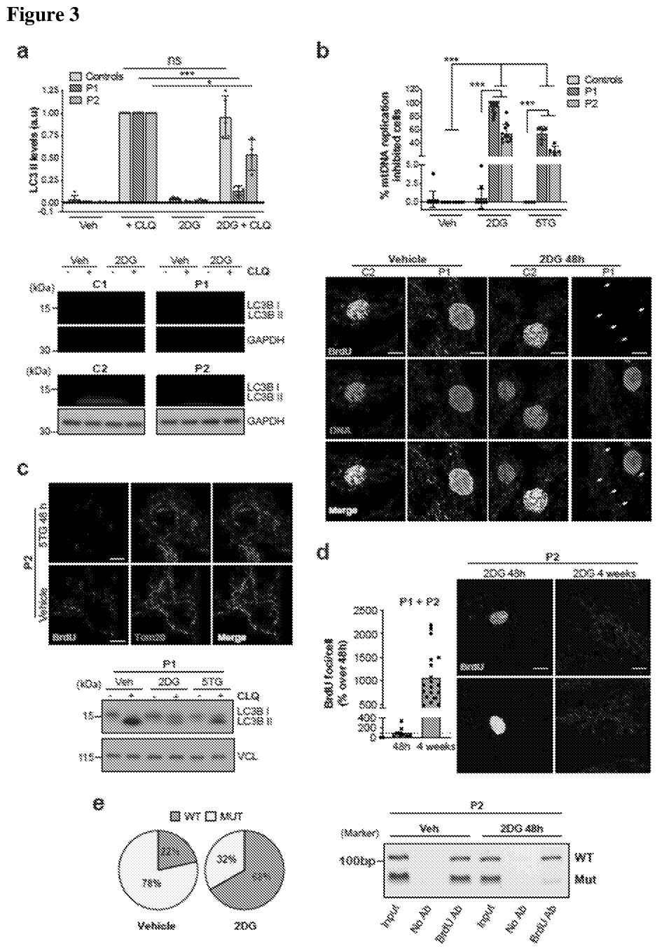

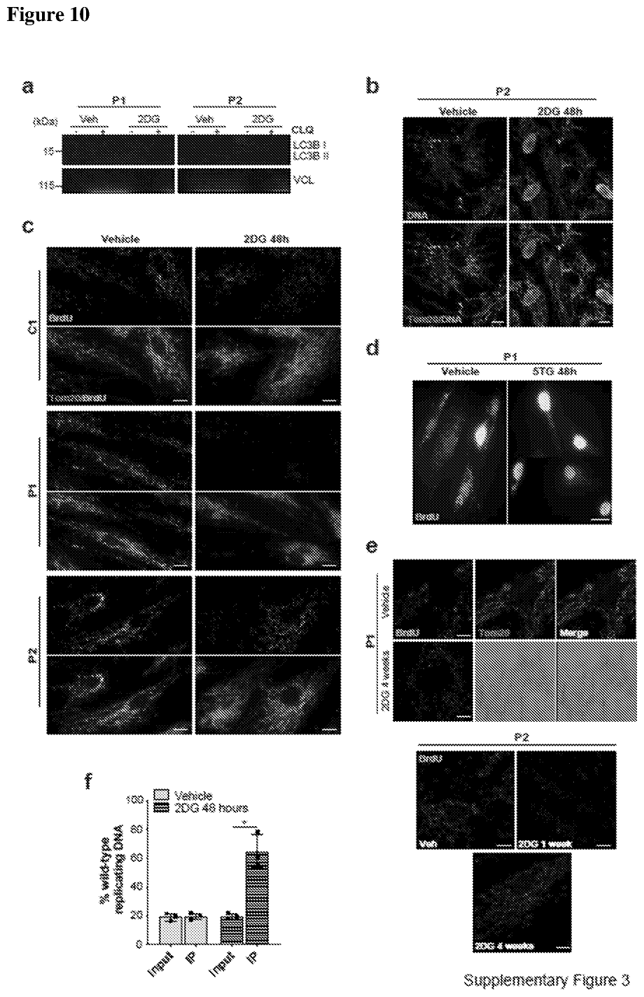

A decrease in the proportion of mutant mtDNA could be achieved by enhanced degradation of m.3243A>G or by selective inhibition of its replication. However, autophagic flux in m.3243A>G cells was repressed, rather than activated, by 2DG, suggesting that mtDNA turnover via autophagy was inhibited in these conditions (FIG. 3a, and FIG. 10a). Furthermore, there was no evidence that 2DG increased mtDNA turnover, based either on copy number measurements or DNA immunostaining (FIG. 3b and FIGS. 8e, 10b). On the other hand, acute 2DG treatment markedly decreased bromodeoxyuridine (BrdU) pulse-labelling of mtDNA in m.3243A>G cells, when compared to the same cells without 2DG or 2DG-treated control fibroblasts (FIG. 3b and FIG. 10c). 5TG also repressed mtDNA replication and autophagic flux in mutant cells, albeit to a lesser extent than 2DG (FIG. 3c and FIG. 10d).

The marked inhibition of mtDNA synthesis in heteroplasmic m.3243A>G cells treated with the glucose analogues strongly suggested that the compounds specifically restricted the replication of the mutant mtDNA. Concordantly, de novo mtDNA synthesis increased by an order of magnitude after four weeks of 2DG treatment in the m.3243A>G fibroblasts (FIG. 3d and FIG. 10e), a period during which the wild-type mtDNA typically increased by a mean of 27 percentage points in the case of 2DG and 37 for 5TG (FIG. 1e-f and FIG. 8c). As further confirmation, the inventors performed a direct test, immunoprecipitating BrdU-labelled DNA from heteroplasmic cells treated with or without 2DG for 48 hours, followed by analysis of the mutant load. While in untreated cells BrdU antibody captured wild-type molecules in a similar proportion to the total mtDNA (1.03:1), in 2DG-treated cells the wild-type mtDNA was enriched 3.25 fold by immunoprecipitation (FIG. 3e and FIG. 10f). This result demonstrated that wild-type mtDNAs have a direct replicative advantage over mutants in the presence of 2DG.

2DG and 5TG De-Energize Cells with Elevated Mutant mtDNA

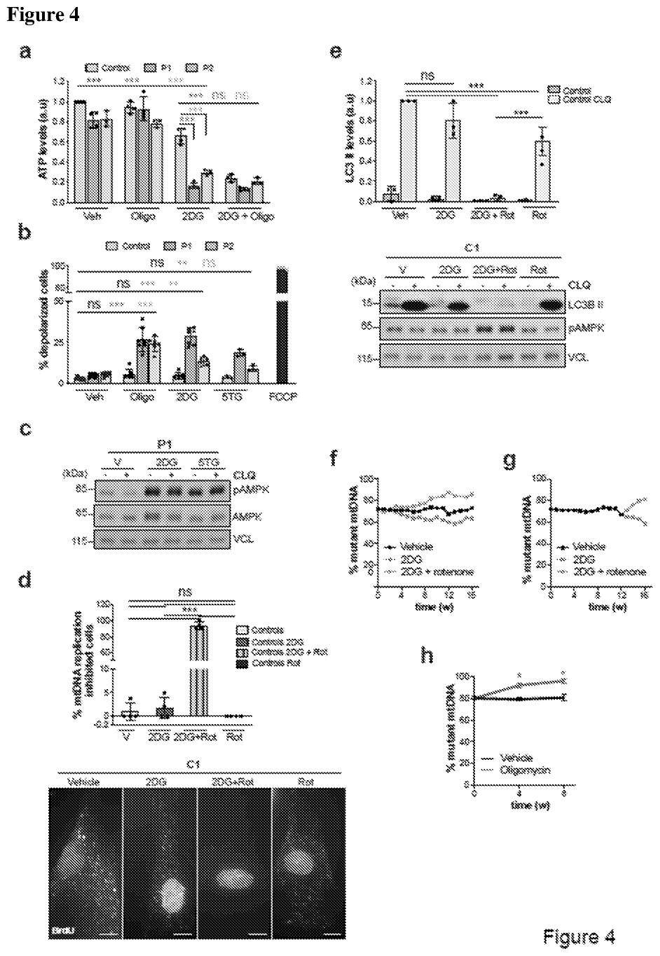

The inventors next determined the impact of 2DG and 5TG on the bioenergetics of the control and cells with mutant mtDNA, via assays of ATP levels and mitochondrial depolarization. Without treatment, when mtDNA replication was not compromised, ATP levels were 80% of control values in patient-derived fibroblasts (FIG. 4a), despite respiration being markedly impaired (FIG. 1i). Nor did inhibition of mitochondrial ATP production with oligomycin significantly affect ATP levels (FIG. 4a); and control cells treated with 2DG maintained their ATP level at 70% of untreated cells. However, 2DG caused a much larger decrease in cellular ATP in m.3243A>G cells than controls. Combining 2DG and oligomycin decreased the ATP level in all the cell lines much more than oligomycin alone (FIG. 4a), thereby demonstrating that 2DG targets an important source of non-mitochondrial ATP, i.e. glycolysis to lactate. On the other hand, 2DG increased the proportion of depolarized mitochondria in control cells from 2% to 5% (FIG. 4b), suggesting that the glucose analogue adversely impacts mitochondria in a small fraction of cells. The effect of 2DG on the mutant cells was considerably greater, as it increased the proportion of cells with depolarized mitochondria from 5% to 25% of the total (FIG. 4b). The dearth of ATP in m.3243A>G cells treated with 2DG was further evidenced by activation of AMP kinase (AMPK), the cells chief energy sensor (FIG. 4c). Together, these data suggested that the compounds caused a severe energy shortage in cells containing m.3243A>G, which could inhibit the replication of the mutant mtDNA limiting its propagation, whereas the mitochondria with some wild-type mtDNA—and thus able to respire—continued to replicate mtDNA.

2DG Impairs mtDNA Replication and Autophagy when Complex I is Inhibited

The inventors inferred that if mitochondrial fitness is important for mtDNA replication, then co-treatment of control cells with 2DG and the complex I inhibitor rotenone should mimic m.3243A>G cells treated with 2DG and inhibit mtDNA synthesis. Accordingly, while rotenone alone had little effect on mtDNA synthesis, the two compounds together inhibited mtDNA synthesis in control cells, greater than, or equal to 2DG in the respiratory deficient m.3243A>G cells (FIG. 4d vs. 3b,d and FIG. 10c,e). The 2DG/rotenone co-treatment of control cells was also equivalent to the 2DG treatment of m.3243A>G cells with respect to inhibition of autophagic flux and AMPK activation (FIG. 4e vs. 3a and FIG. 10a). These data indicated that 2DG forces control cells to depend on mitochondrial energy production for mtDNA replication and autophagy. The findings also explained how 2DG has a greater impact on mitochondria with m.3243A>G than those with wild-type mtDNA in heteroplasmic cells: the mutant mitochondria are complex I deficient and so equivalent to mitochondria of control cells treated with 2DG and rotenone, whereas replication should remain active in the few mitochondria with wild-type mtDNA, as they possess a functional respiratory chain.

Thus, the wild-type mtDNA derives its selective advantage over m.3243A>G from the fact that replication becomes respiration/complex I-dependent in the presence of 2DG. Combined rotenone and 2DG treatment should negate any selective advantage of wild-type mtDNA conferred by 2DG in heteroplasmic m.3243A>G cells, as should other inhibitors of mitochondrial energy production. The primary fibroblasts carrying m.3243A>G did not survive long-term treatments with OXPHOS inhibitors; however, in A549 cells with m.3243A>G, rotenone with 2DG reversed the direction of mtDNA segregation, compared to 2DG alone (FIG. 4f,g); and oligomycin-induced inhibition of ATP synthase also promoted segregation to mutant mtDNA on the A549 background (FIG. 4h). Negation of mitochondrial fitness by the OXPHOS inhibitors will permit the selection of the mutant mtDNA by selfish mechanisms, such as decreased replication pausing.

Cells with Mutant mtDNA are More Reliant on Glutamine for mtDNA Replication than Controls

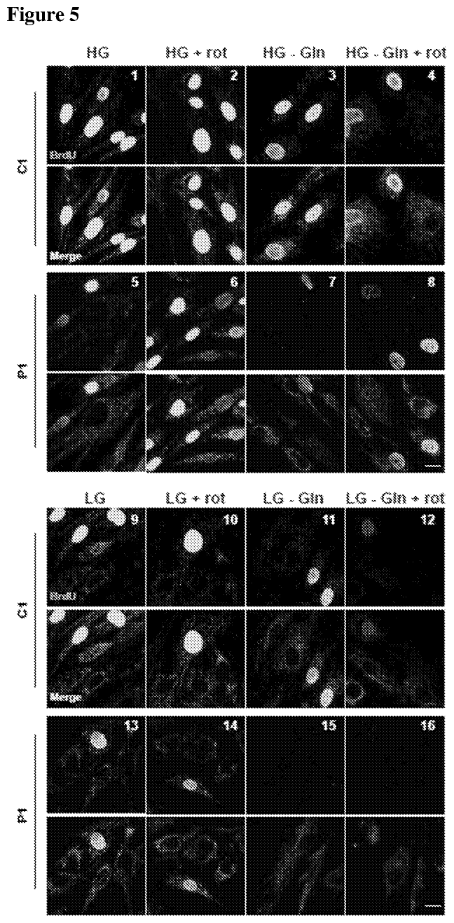

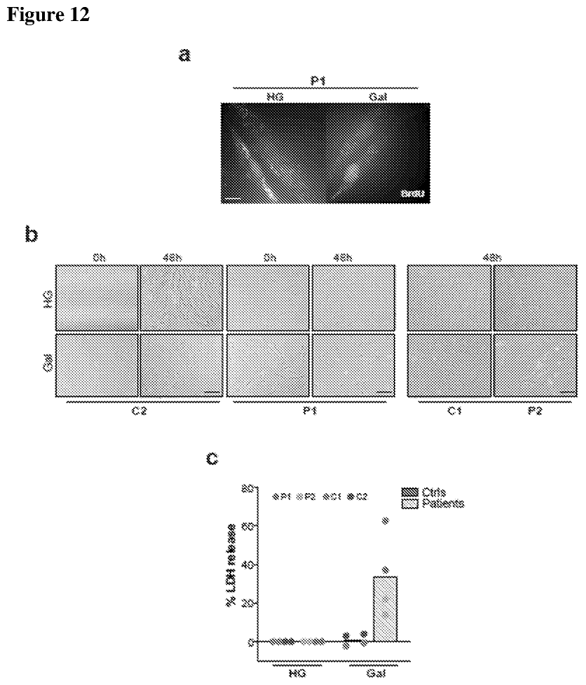

Although glucose metabolism is the most obvious target of 2DG to affect cellular bioenergetics and mitochondrial fitness, the compound also inhibits glutamine utilization; a process that could provide critical support to the replication of mutant mtDNA, given that cells with mitochondrial dysfunction are heavily reliant on glutamine. Therefore, we assessed the contributions of glucose and glutamine to mtDNA replication by restricting their availability, adding rotenone in some experiments as a ‘m.3243A>G mimetic’. Glutamine withdrawal inhibited mtDNA replication in the cells with a high mutant load, much more than in control cells (FIG. 5, panels 1 vs. 3 (control) and 5 vs. 7 (m.3243A>G)), demonstrating that the increased glutamine consumption associated with mitochondrial dysfunction is supporting mtDNA replication. Moreover, as neither glucose restriction (FIG. 5, panel 13), nor replacement of glucose with galactose (prior to cell death) (FIG. 12), inhibited mtDNA synthesis in mutant cells, the inventors concluded that restricting glutamine utilization is the means by which 2DG inhibits the replication of mutant mtDNA.

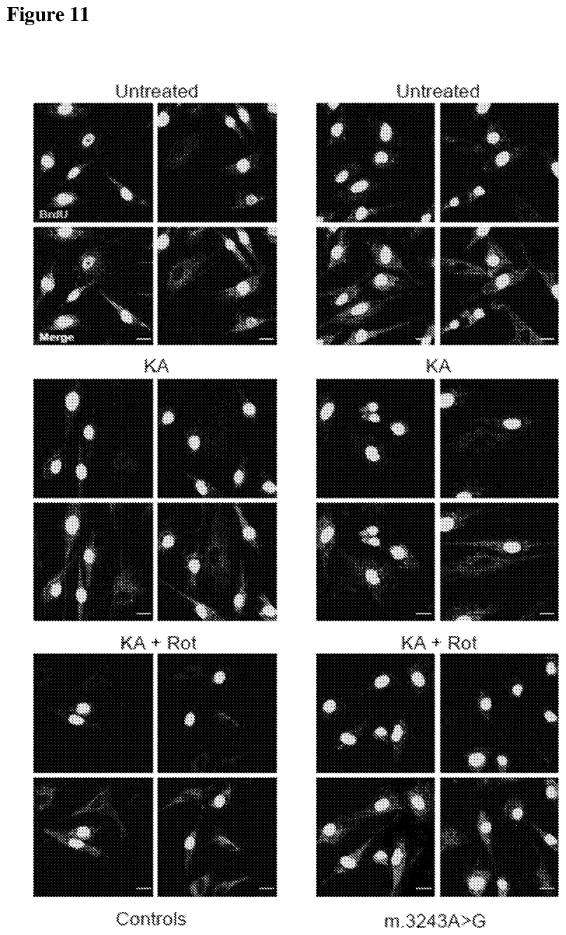

Notwithstanding the above, further analyses indicated that glucose is critical for mtDNA replication in some contexts, and that rotenone does not model all the features of m.3243A>G. While the 2DG/rotenone co-treatment inhibited mtDNA replication in controls similar to 2DG in mutant cells (FIG. 4d), glutamine withdrawal and rotenone treatment in controls (FIG. 5 panel 4) did not mimic the effect of glutamine deprivation observed in mutants (FIG. 5 panel 7). Instead, it was necessary to restrict glucose (to 1 mM), as well as to add rotenone and withdraw glutamine, to block replication in control cells (FIG. 5 panel 12). Evidently, the 1 mM glucose is utilized by Complex I to support mtDNA replication, given that mtDNA synthesis is active when both glucose and glutamine are restricted unless respiration is inhibited with rotenone (FIG. 5 panel 11 vs. panel 12). Nevertheless, glycolysis and complex I are dispensable for mtDNA synthesis. Not only was replication maintained upon rotenone treatment in 1 mM glucose in both control and mutant cells (FIG. 5.10), it was also active when the cells were treated with the glycolytic (GAPDH) inhibitor Koningic acid (KA), with and without rotenone (FIG. 11).

This web of comparisons distils down to the conclusion that cells have three means of sustaining mtDNA replication: 1) glycolysis; 2) glucose-supported respiration via complex I; and 3) glutamine metabolism. Their respective contributions are not fixed but vary according to metabolite availability and pathway flux; i.e. they are in a state of dynamic equilibrium. It then becomes evident that complex I deficiency, by increasing glycolytic ATP production and elevating glutamine consumption in the mutant cells, will make the replication of their mtDNA susceptible to changes in glucose and glutamine distinct from control cells. These distinctions, particularly as regards complex I activity, will apply equally at the intracellular level, where there is a mixture of mitochondria with mutant and wild-type mtDNA in the heteroplasmic state. In conclusion, mtDNA replication is dependent on nutrient availability and mitochondrial (dys)function.

Combined Glutamine and Glucose Restriction Promotes the Selection of Wild-Type mtDNA Equal to 2DG

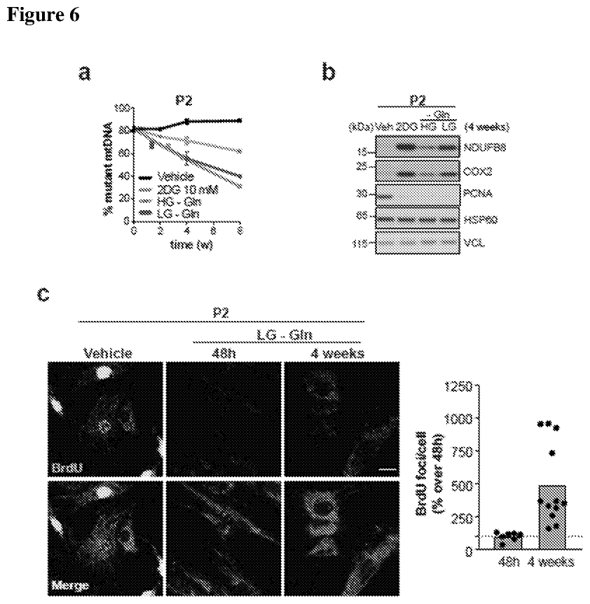

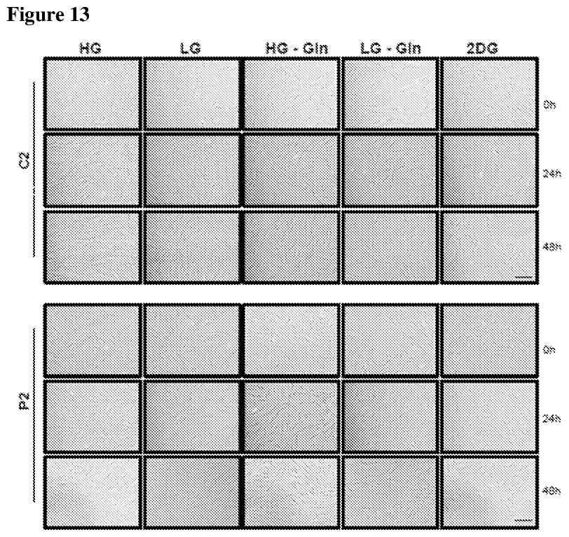

Because 2DG limits the utilization of glutamine and glucose, the inventors next investigated their effects on the mtDNA selection directly. The inventors maintained m.3243A>G heteroplasmic cells in four conditions: standard medium containing 25 mM glucose, 4 mM glutamine; the same medium but without glutamine, or media with 1 mM glucose with and without glutamine. Mutant cells in 1 mM glucose with glutamine grew more slowly than control cells (FIG. 13a), which would drive selection via intercellular competition; additionally, they started dying in substantial numbers after three pulses of this medium. Thus, the specific effect of glucose restriction on mtDNA segregation could not be assessed. In contrast, the combined glutamine and glucose restriction regime slowed growth equally in control and mutant cells (FIG. 13), as per 2DG (FIG. 9c and FIG. 13), and was not associated with cell death, indicating that the absence of glutamine enhances mutant cell survival when glucose is limiting. While glutamine withdrawal produced a modest increase in the level of wild-type mtDNA, it was the combination of glutamine and glucose restriction that recapitulated the effect of 2DG on segregation (FIG. 6a), leading to increases in OXPHOS subunits levels and mtDNA synthesis (FIG. 6b,c). Taken together, the data suggested that while mutants were more dependent on glutamine, functional wild-type mtDNA were more reliant on complex I-dependent energy production (when glucose is scarce) and could use this to maintain the mitochondrial membrane potential. Therefore, the inventors inferred that combined glutamine and glucose restriction imposes twin selective pressures—negative on the mutant and positive on wild-type mtDNAs—and that that 2DG is effective at driving segregation to wild-type molecules because it restricts the utilization of both substrates.

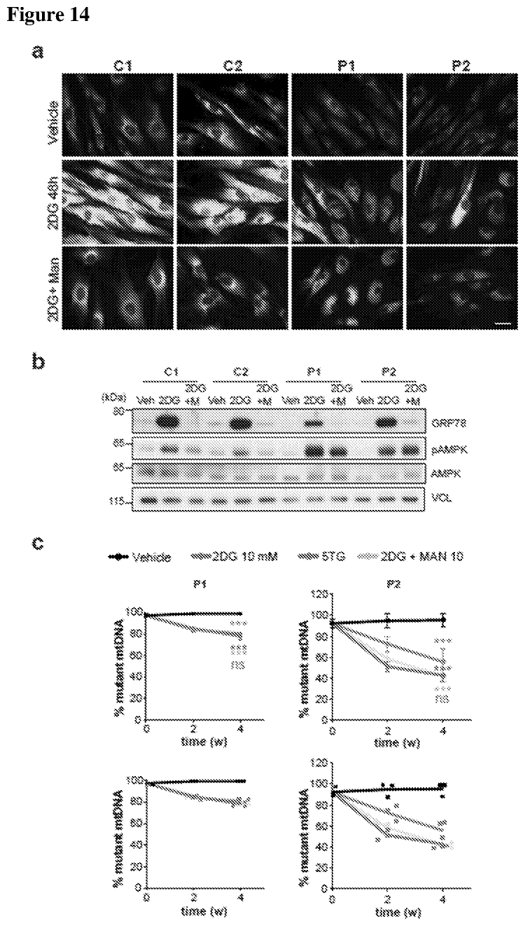

As well as inhibiting glycolysis and restricting glutamine utilization, 2DG induces ER-stress, as it is structurally similar to mannose. The inventors confirmed that 2DG increased GRP78 expression and that this was attenuated by mannose, without inactivating AMP kinase (FIG. 14a,b). Nevertheless, 2DG with mannose was at least as effective at inducing segregation to wild-type mtDNA as 2DG alone (FIG. 14c,d). Therefore, 2DG's effect on mtDNA segregation does not relate to its similarity to mannose, nor GRP78-related ER-stress.

### Discussion

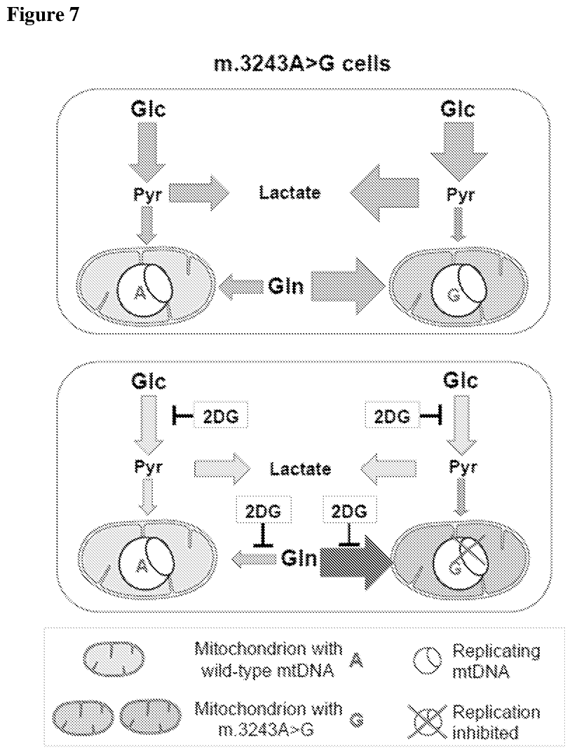

This study has identified in 2DG, and the related 5TG, small molecules that can purge cells of the common pathological mtDNA variant m.3243A>G, restoring mitochondrial respiratory capacity. The inventors' analysis of the mechanism of action indicated that 2DG preferentially depolarizes mutant mitochondria, inhibits the replication of mutant mtDNA and allows the propagation of functional mtDNA molecules. Moreover, as the inventors show that restricting glutamine and glucose recapitulates the effects of 2DG on mtDNA replication and segregation, the inventors conclude that both glucose and glutamine metabolism are critical targets of 2DG (FIG. 7).

Logically, the positive selection of wild-type mtDNA depends ultimately on its capacity to produce functional products for OXPHOS. Given that mtDNA replication is dependent on respiration via complex I when control cells are exposed to 2DG (FIG. 4d), or glutamine and glucose are restricted (FIG. 5 panel 11), it is respiration via complex I that gives the mitochondria with wild-type mtDNA their selective advantage. Oppositely, inhibition of OXPHOS reverses segregation to wild-type mtDNA (FIG. 4f-h), again indicating that mitochondrial fitness is essential for the maintenance and selection of functional molecules. That said, the demonstration that cells/mitochondria with high levels of m.3243A>G are replication-competent when glycolysis is reduced (FIG. 5 panel 13 and FIGS. 11-12) potentially explains how deleterious mtDNA variants of this type are transmitted in the human germline: mitochondria with m.3243A>G need not depend on glycolysis for mtDNA maintenance, and so the desired selective pressure might be lacking, if, for example, glutamine is readily available.

The identification of small molecules that promote the selection of wild-type mtDNA over mutant m.3243A>G—restoring mitochondrial protein synthesis and respiratory capacity—advances the prospect of pharmacological treatments for heteroplasmic mtDNA disorders. Moreover, increased glutamate utilization is a feature of mitochondrial dysfunction in a mouse model, and the predominant complex I deficiency associated with m.3243A>G mtDNA in cells is also seen in muscle of subjects with this mutant. Thus, the identified mechanism of action of 2DG is expected to be applicable in vivo; that is, the mutant mitochondria in vivo will also be more reliant on glutamine utilization than their wild-type counterparts, while the latter benefit from much higher complex I capacity. Furthermore, the imposition of mitochondrial fitness selects against defective mtDNAs in flies and in the mammalian germline the selection of functional mitochondria coincides with a switch from glycolytic to oxidative metabolism.

Although 5TG appears to be as effective as 2DG at positively selecting wild-type mtDNA, and it does not induce the pronounced ER-stress of 2DG, it has never been tested in humans. Mannose, on the other hand, is an approved dietary supplement, which depresses the ER stress caused by 2DG (FIG. 14a,b) without interfering in the selection of wild-type mtDNAs (FIG. 14c). Therefore, the combination of 2DG and mannose may well be the best prospect for treatment of m.3243A>G and other heteroplasmic mtDNA disorders than 2DG alone.

### Studies in Mice

An in vivo study of 2DG in healthy control mice demonstrated that 2DG increases the abundance of the respiratory chain (RC) in the murine heart after 6 months of intermittent treatment. The abundance of the RC is a fundamental measure of mitochondrial ATP capacity and demand for mitochondrial energy production (tissues and organs with a high energy demand have the highest RC levels). The weekly dose used was lower than that used in human cancer trials. Therefore, the mouse study indicates that 2DG is pharmacologically active in vivo at a dose that can be used in humans, and it indicates that 2DG promotes the propagation of functional mitochondria/mtDNAs in vivo as well as in cells cultured in the laboratory.

### Use of Oxamate

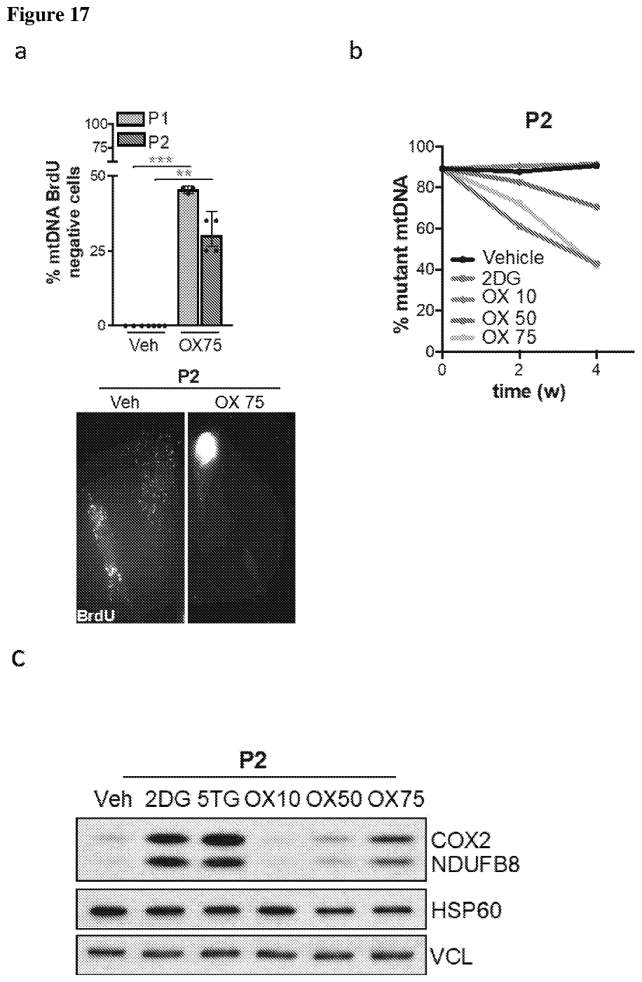

It has also been shown that oxamate can be used to inhibit mtDNA replication in cells carrying high levels of mutant mtDNA. In addition, oxamate selects wild-type mtDNA in heteroplasmic cells. The decrease in mutant load increases the levels of OXPHOS subunits after four weeks of treatment. See FIG. 17. Oxamate limits the conversion of glucose to lactate via glycolysis among other effects.

### Materials and Methods

### Cell Culture

A549 adenocarcinoma and MyoRD rhabdiomyosarcoma m.3243A>G cybrid cells (Dunbar, D. R. et al., Proc Natl Acad Sci USA 92, 6562-6566 (1995) and Malena, A. et al., Autophagy 12, 2098-2112 (2016)) were maintained in Dulbecco's Modified Eagle's Medium (DMEM) containing 25 mM glucose (Life Technologies) supplemented with 10% fetal bovine serum (FBS, Pan Biotech UK), 1 mM of pyruvate, 1% penicillin and streptomycin (PS, Life Technologies), at 37° C. in a 5% C02 atmosphere. Primary skin fibroblasts were grown in DMEM GlutaMAX™ (Life Technologies) with the same supplements. All the cell lines were regularly confirmed free of mycoplasma, using the Look Out Mycoplasma PCR Detection Kit (Sigma).

Glucose restriction employed glucose-free DMEM medium (Life Technologies) with the addition of no or 1 mM glucose, as indicated, whereas galactose was added to 5 mM, plus 10% dialyzed or non-dialyzed serum, also as indicated. For the glutamine restriction experiments, 1 mM or 25 mM glucose was added to DMEM lacking glutamine, supplemented with 10% dialyzed serum.

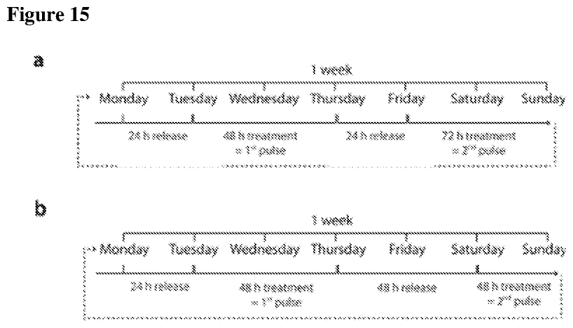

For the acute treatments, cells carrying m.3243A>G were grown to 50-60% confluent and treated for 24 or 48 hours with the compounds and concentrations indicated in the main text, figures and methods. Intermittent treatments extending over several weeks comprised 48 or 72 h pulses with the drug or modified medium, followed by 24 or 48 h of recovery, throughout the course of the experiments (see FIG. 15).

### Inhibitors and Chemicals

Chemicals were purchased from Sigma, except for Koningic acid (Abcam). Rotenone, oligomycin and 5-bromo-2′-deoxyuridine were dissolved in DMSO, whereas all the other chemicals were dissolved in milliQ-grade water. The final concentrations were as follows: 10 mM 2-deoxyglucose (unless otherwise specified), 10 mM 5-thioglucose, 10 mM Mannose, 0.5 or 1 μM Rotenone, 50 μM Chloroquine, 50 μM 5-bromo-2′-deoxyuridine mM, 1 μM Oligomycin, 0.5 or 1 μM Koningic acid.

### Cellular Proliferation Rate and Vitality

The cellular proliferation rate was determined using an IncuCyte Zoom cell imager (Essen Bioscience). 3×10⁴ cells were seeded in 6-well plates and imaged every hour for 3 d. The proliferation rate was determined using the Incucyte Zoom software 2015A. At the end of the treatment, the cells were labelled with 5 μM calcein (Molecular Probes, Thermo Fisher Scientific) for 20 minutes and then imaged.

### Cytotoxicity Assay

The release of the Lactate Dehydrogenase (LDH) in the medium was measured adapting the instructions from the manufacturer (Cytotoxicity Detection Kit, Roche). Briefly, control and patient fibroblasts were seeded on multi-six-well plates (Thermo Fisher Scientific) and subjected to either vehicle or 2DG treatment for 48 h or cell grown in galactose medium. Cells were seeded at different densities, taking in account the differences in their growth rate: 3×10⁴ for vehicles and 6×10⁴ for 2DG—treated cells. For the positive control, cells were treated with 1% Triton X-100 (Santa Cruz Biotechnology). 100 μL of the medium was used for each assay. After incubating the medium with the dye for 30 minutes at room temperature, the absorbance at 490 nM was measured using a plate reader (Biorad). The data were then normalised for the protein content after cell lysis.

### Fractionation and Immuno-Detection of Proteins

Cells were lysed on ice with RIPA buffer (65 mM Tris, 150 mM NaCl, 1% Nonidet P-40), 0.25% Na-DOC, 1 mM EDTA, pH 7.4) 1× protease inhibitor cocktail (PIC, Roche), phosphatase inhibitor cocktail (Cell Signalling), 50 U Benzonase (Millipore). After incubating on ice for 20 minutes, the samples were centrifuged for 20 minutes at 13000 g, to separate the proteins from the DNA. Protein concentration was determined by DC protein assay kit (Biorad). Protein samples were prepared in 1× Laemmli loading buffer and resolved on 4-12% or 10% or 12% Bis-Tris NuPAGE gels (Life Technologies, Thermo Fisher Scientific) run in NuPAGE MES or MOPS buffers (Life technologies, Thermo Fisher Scientific). After electrophoresis, proteins were transferred to a polyvinylidene fluoride membrane (PVDF, Millipore) and blocked in 5% milk (Sigma), PBS containing 0.1% Tween (Thermo Fisher Scientific) for 1 h. Membranes were incubated overnight with primary antibodies (see below), at 4° C. and, after washes, with the appropriate secondary antibodies for 1 h at room temperature. Proteins were detected using standard ECL™ Western Blotting Analysis System (GE Healthcare) or SuperSignal®West Dura (Thermo Scientific). Western blots were digitalized using a Canoscan 9000F scanner (Canon). Optical density quantification of bands detected by Western blotting was carried out using the designated tools available with Fiji ImageJ (2.0.0-rc-15/1.49 h).

### Antibodies

The following primary antibodies were used in this study: BrdU (Biorad, MCA2060 or Abcam, ab6326, 1:200 dilution); DNA (Progen, AC-30-10, 1:250 dilution); GAPDH (Sigma, G8795 or Abcam, ab8245, 1:10000 and 1:2000 dilutions, respectively); GRP78 (Santa Cruz Biotech, Sc-13968, 1:1000 dilution); HSP60 (Abcam, ab46798, 1:1000 dilution); LC3B (Sigma, L7543) 1:5000; MTCO-2 (Abcam, ab110258 1:1000 dilution); NDUFB8 (Abcam, ab110242, 1:1000 dilution); AMPK alpha (Cell Signaling, 2532, 1:1000 dilution); phosphoAMPK alpha (Cell Signaling, 2531, 1:1000 dilution); TOM20 (Santa Cruz Biotech or Abcam, Ab186735, 1:4000 and 1:10000 dilutions, respectively); VCL (Abcam, ab18058, 1:1000 dilution); PCNA (Mouse, sc-56, 1:8000 dilution).

Secondary Antibodies: Anti-Mouse IgG (H+L), HRP Conjugate (Promega, W4021, 1:4000 dilution); anti-Rabbit IgG (H+L), HRP Conjugate (Promega, W4011 1:4000 dilution); Alexa Fluor®-488 goat-anti-mouse (Invitrogen, A-10684, 1:1000 dilution); Alexa Fluor®-568 goat-anti-mouse (Invitrogen, A-11004, 1:1000 dilution); Alexa Fluor@ 568 donkey anti-rabbit (Invitrogen, A-10042, 1:1000 dilution); Alexa Fluor®-488 goat-anti-rat (Invitrogen, A-11006, 1:1000 dilution).

### Determination of Mutant Load

DNA was extracted from cells using the Puregene system (Qiagen) or Wizard SV Genomic DNA Purification System (Promega), and the proportion of wild-type mtDNA and m.3243A>G was determined by pyrosequencing, which has been validated for quantification of m.3243A>G heteroplasmy (White, H. E. et al., Genetic testing 9, 190-199 (2005)). Briefly, a 155 base pair region of human mtDNA encompassing the m.3243A>G site was amplified using the PyroMark PCR kit (Qiagen). Pyrosequencing reactions were performed using a sequencing primer and PyroMark reagents (Qiagen) on a PSQ 96MA pyrosequencer and analysed with PSQ 96MA 2.1 software. Pyrosequencing exhibited a standard deviation range of 0.06-4.64% change in heteroplasmy across 359 samples measured in triplicate. Last-cycle PCR of sequence spanning bp 1155-1725 of human mtDNA that includes an invariant ApaI site was used as a positive control to confirm complete digestion. Alternatively, heteroplasmy was measured by restriction fragment length polymorphism analysis, using amplified mtDNA spanning bp 2966-3572; and the mutant load was estimated from the proportion of DNA cleaved by ApaI, after separation of digested PCR product via agarose gel electrophoresis (Turner, C. J. et al., Genetics 170, 1879-1885 (2005)).

### BrdU-DNA Immunoprecipitation

Primary skin fibroblasts treated acutely with 10 mM 2DG received a 16 h pulse of 50 μM bromo-deoxyuridine (BrdU, Sigma). Parallel conditions without BrdU and/or without 2DG were used as controls. One microgram of isolated DNA was digested with ApoI restriction enzyme to generate −300 bp fragments containing the m.3243A>G site. Fragmented DNA was then denatured for 10 min at 95° C. in PBS containing 10 μg of sheared Salmon Sperm DNA (Invitrogen) in a final volume of 50 μl. Samples were precleared with 50 μl of 50% Protein G Agarose beads solution (Thermo Scientific) for 2 h at 4° C. with constant shaking. Beads were pelleted by centrifugation and the supernatant incubated overnight at 4° C. with 1 μg of anti-BrdU primary antibody in 200 μl of PBS containing 0.625% triton X-100. Antibody-DNA complexes were captured with 50 μl of 50% Protein G Agarose beads solution (Thermo Scientific) for 1 h at 4° C. Beads were then washed 3×5 min with 1% Triton X-100, 0.1% SDS, 150 mM NaCl and 2 mM EDTA in 20 mM Tris pH 8.0 followed by a final wash with 1% Triton-X100, 0.1% SDS, 500 mM NaCl, 2 mM EDTA in 20 mM Tris pH 8.0. DNA was eluted with 1% SDS in TE buffer for 15 min at 65° C. For each sample, a second tube without antibody incubation was run in parallel as control. Eluates form each sample were purified with phenol:chloroform and resuspended in 20 μL TE buffer. The mutant load in the immunoprecipitated DNA was estimated by restriction fragment length polymorphism analysis of amplified mtDNA spanning bp 3202-3328 (see—Determination of mutant load above).

Quantification of the mtDNA Copy Number

The mtDNA copy number was quantified as follows: after DNA isolation, real-time quantitative PCR was performed in triplicates on 384-Well Reaction Plates (Applied Biosystems) in final volumes of 10 μL. Each reaction contained 20 ng of DNA template, 1× Power SYBR-Green PCR Master Mix (Applied Biosystems) and 0.5 μM of forward and reverse primers. Mitochondrial and nuclear DNA were amplified using primers specific to regions of human COX2 and APP1 genes. Changes in the mtDNA copy number were determined by using the 2-ΔΔCt method and represented as fold-change relative to the mean value for vehicle-treated cells analysed in parallel (Dalla Rosa, I. et al., PLoS Genet 12, e1005779 (2016)).

### Mitochondrial Translation Assay

Mitochondrial translation products were labelled using 35S-methionine (Durigon, R. et al., EMBO Mol Med 10(9): e8550 (2018)). Fibroblasts were washed twice with methionine/cysteine free DMEM (Life Technologies) supplemented with 1 mM L-glutamax, 96 μg/ml cysteine (Sigma), 1 mM pyruvate and 5% (v/v) dialyzed FBS, and incubated in the same medium for 10 min at 37° C. 100 μg/ml emetine dihydrochloride (Sigma) was added to inhibit cytosolic translation, before pulse-labelling with 100 μCi [35S]-methionine for 45-60 minutes. Cells were chased for 10 min at 37° C. in regular DMEM with 10% FBS, washed three times with PBS and harvested. Labelled cells were lysed in PBS, 0.1% n-dodecyl-D-maltoside (DDM), 1% SDS, 50 U Benzonase (Millipore), 1× protease inhibitor cocktail (Roche). Protein concentration was measured by DC protein assay kit (Biorad) and 20 μg of protein were separated by 12% SDS-PAGE. The gels were then stained with the coomassie staining solution (50% Methanol (Fisher Scientific), 10% Acetic Acid (Sigma), 0.1% Coomassie Brilliant Blue R250 (Biorad)) to confirm equal loading. Gels were then dried and exposed to phosphor screens (GE Healthcare). The signal was detected by using Typhoon™ Phosphoimager (GE Healthcare).

### Oxygen Consumption Rate (OCR) and Extracellular Acidification Rate (ECAR) Measurements

Mitochondrial respiration was assayed in fibroblasts treated or not with 2DG on 24 wells XF24e plates, using an XF24e Extracellular Flux Analyzer (Agilent Technologies). Briefly 5×10⁴ cells were seeded approximately 16 hours before the assay in pre-warmed growth medium (DMEM, GIBCO) and incubated at 37° C. Subsequently, the medium was removed and replaced with assay medium (XFBase medium minimal DMEM (Agilent) complemented with 2 mM glucose, 2 mM glutamax and 1 mM pyruvate) and cells incubated for 30 min in a 37° C. non-CO₂ incubator. After taking an OCR baseline measurement, 1 μM oligomycin, 0.75 μM carbonylcyanide-4-trifluorometho-xyphenylhydrazone (FCCP) and 1 μM rotenone were added sequentially. For the ECAR values, the average of the first three measurements of the basal level prior the oligomycin injection was considered.

### Immunofluorescence and DNA Labelling

Control and patients' fibroblasts were grown on chamber slides (Thermo Fisher Scientific) and fixed with 4% formaldehyde (Sigma) in phosphate-buffered saline (Sigma) for 20 minutes at 37° C. After washing, the cells were permeabilised with 0.3% Triton X-100 (Santa Cruz Biotechnologies) in PBS containing 5% FBS. For the bromo-deoxyuridine (BrdU, Sigma) incorporation experiment, the cells were incubated with BrdU 50 mM for 13-16 h, then fixed, permeabilised and treated with HCl 2N for 20 min at 37° C. Cells were then blocked with PBS containing 5% FBS and incubated with primary antibody overnight at 4° C. After washes, slides were incubated with the appropriate secondary antibody for 1 hour at room temperature. Slides were then washed and mounted over ProLong® Gold Antifade Reagent (Thermo Fisher Scientific) without DAPI nuclear staining.

### Image Capture and Analysis

Samples were imaged either on a Leica SP5 TCS Inverted Confocal Microscope or a Nikon Ti Inverted Confocal microscope. The microscope software for Leica was Leica Application Suite X, with the file extension “.lif” format, whereas for the Nikon microscope we used NIS Element Software in a “.nd2” format. Z stack of red, green and blue images was acquired sequentially and merged using Image J. Laser power, gain and offset parameters were kept constant for each experiment. The image analysis was performed using the plugins available in Fiji imageJ (2.0.0-rc-15/1.49h); any adjustments to brightness and contrast were applied linearly to all images in a comparison. Treated cells with far fewer BrdU positive foci than the corresponding untreated cells, such as those shown in 2b-2d, were scored ‘inhibited for mtDNA replication’.

### ATP Measurements

Total intracellular ATP levels were measured by bio-luminescence using a luciferin-luciferase system according to the manufacturer's instructions. Cells were plated in duplicate 24 well plates, and treated the following day with 10 mM 2DG or 1 μM oligomycin alone for 24 hours, and 10 mM 2DG for 24 hours with addition of 1 μM of oligomycin for the last 4 hours. One plate was used to determine the total protein amount of samples, and the luminescence signal was normalized to the total amount of protein.

### Mitochondrial Membrane Potential

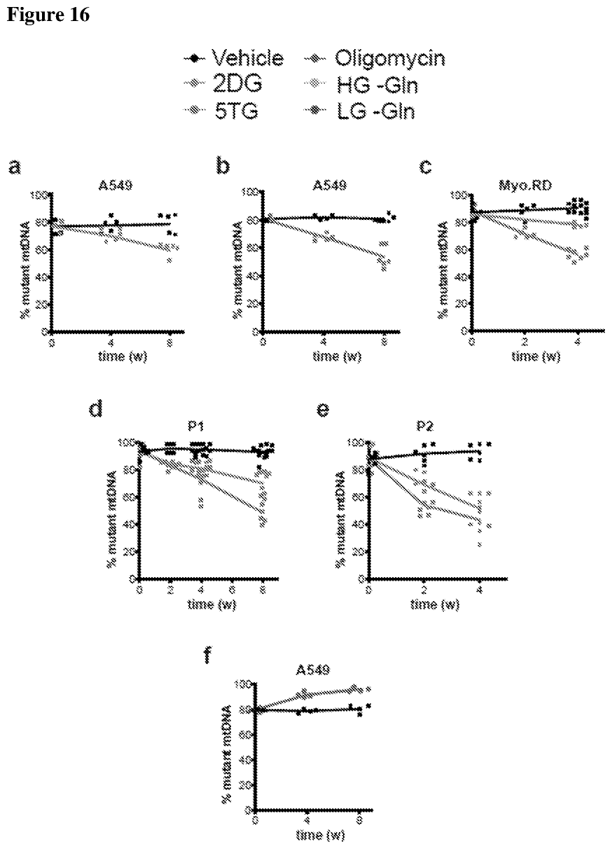

Mitochondrial depolarization was evaluated by measuring the loss of TMRM (tetramethylrhodamine methyl ester; Molecular Probes Thermofisher Scientific, T668) staining by FACS analysis in non-quenching mode (FACS Analyzer LSRFortessa 5 laser SORP, Becton-Dickinson, Diva Software version 8). Gating strategy is illustrated in FIG. 16). Cells were seeded in 12 well plates, treated with 10 mM 2DG or 5TG for 24 hours and incubated with 20 nM TMRM and 1.6 μM cyclosporine H (Enzo Life Sciences, ALX-380-286) for 30 min. Cells were washed with PBS, trypsinised, centrifuged, resuspended in 300 μl of PBS and acquired. 1.6 μM oligomycin was added in the test tube to a set of separate untreated samples for 90 min and then acquired. Physical parameters were used to gate the singly dispersed cells. Addition of 4 μM of FCCP (carbonyl cyanide 4-(trifluoromethoxy)phenylhydrazone, Sigma) on untreated samples was used to completely depolarize the mitochondria at the end of the experiment.

### Rate of Shift of Heteroplasmy

To compute heteroplasmy shifts beta between initial heteroplasmy h0 and final heteroplasmy h after time t, we used the formula beta t=log((h(h0-1))/(h0(h−1))) from (8). We used the z-test against a null hypothesis of zero mean to compute P-values.

### Statistics Analysis

Data were collated in Excel 14.4.8. Statistical analyses were performed using Graphpad Prism (v.7 and 8). Immunoblots and mitochondrial protein synthesis were analysed with Fiji ImageJ. Data were expressed as mean values±standard deviation (SD) or standard error of mean (SEM). Data were analysed using a two-sided non-parametric Mann-Whitney U test, a two-sided parametric t-test, a two-sided one-sample median test and a two-sided Wilcoxon signed-rank test. Multiple comparisons were performed with one-way ANOVA test. Comparisons were considered statistically significant for P values<0.05 (*P<0.05, ** P<0.005, ***P<0.001); ns—not significant. Exact P values are reported in the figure legends. The number of replicates for each independent experiment is stated in the corresponding figure legend.

1. A method of treating a mitochondrial DNA disorder, comprising administering a therapeutically effective amount of a compounds to a patient suffering from a mitochondrial DNA disorder, wherein the compound is a glycolysis inhibitor, an inhibitor of glutamine consumption, L-asparaginase, or pegaspargase, thereby treating the mitochondrial DNA disorder in the patient.

2. The method according to claim 1, wherein the compound is the glycolysis inhibitor, L-asparaginase, or pegaspargase.

3. The method according to claim 1, wherein the compound is the glycolysis inhibitor.

4. The method according to claim 1, wherein the compound is a glucose analogue.

5. The method according to claim 4, wherein the glucose analogue is 2-deoxy-D-glucose (2DG), 2-fluoro-2-deoxy-d-glucose (2-FG), 2-chloro-2-deoxy-d-glucose (2-CG), 2-bromo-2-deoxy-d-glucose (2-BG), 5-thioglucose (5TG), 2-fluoro-d-mannose (2-FM), acetyl 2-DG analogues, 1,5 anhydro-D-fructose, 6-O benzyl-D-galactose, C3361 or 3,6-di-O-acetyl-2-deoxy-d-glucose.

6. The method according to claim 5, wherein the glucose analogue 2-deoxy-D-glucose (2DG) or 5-thioglucose (5TG).

7. The method according to claim 6, wherein the glucose analogue is 2DG.

8. The method according to claim 6, wherein the glucose analogue is 5TG.

9. The method according to claim 1, wherein the compound is oxamic acid or oxamate.

10. The method according to claim 1, wherein the compound is L-asparaginase or pegaspargase.

11. The method according to claim 1, wherein the compound is L-asparaginase.

12. The method according to claim 1, wherein the compound is the inhibitor of glutamine consumption.

13. The method according to claim 1, wherein the compound is an inhibitor of an enzyme, and wherein the enzyme is a glucose transporter, hexokinase, glucose-6-phosphate dehydrogenase, transketolase, phosphoglucose isomerase, phosphofructokinase, aldolase, triosephosphate isomerase, glyceraldehyde-3-phosphate dehydrogenase, phosphoglycerate kinase, phosphoglycerate mutase, enolase, pyruvate kinase, lactate dehydrogenase or a lactate transporter.

14. The method according to claim 1, wherein the compound is Phloretin, Quercetin, Fasentin, STF31, WZB117, 3-bromopyruvic acid, 3-bromopyruvate, D-Mannoheptulose, N-acetylglucosamine, Imatinib, Lonidamine, SID 856002 (Ebselen), SID 17387000, SID 24785302, SID 3716597, SID 24830882, SID 16952891, SID 22401406, SID 24797131, SID 17386310, SID 14728414, 6-aminonicotinamide (6AN), dehydroepiandrosterone (DHEA), oxythiamine chloride hydrochloride, p-hydroxyphenylpyruvate, diphenylurea derivatives T2, T2A, T2B, T2C, T2D and T2E, d-arabinose-5-phosphate derivatives, ST090269, ST082230, ST078079, 5251606, 7993994, 6877084, ST060239, 7963836, 6125285, 5150036, 7950244, 9064882, 5116964, 5224468, 9074873, 9193149, 5331342, 7745039, ST093058, ST057360, 3-(3-pyridinyl)-1-(4-pyridinyl)-2-propen-1-one (3PO), PFK158, Auranofin, N4A,5,6,7,8-tetrahydroxy-2-(4-hydroxyphenyl) chromen-4-one, 7,8-dihydroxy-3-(4-hydroxyphenyl) chromen-4-one), ethyl 7-hydroxy-2-oxochromene-3-carboxylate, bisphosphonate inhibitors, naphthalene 2,6-bisphosphate, 2,6-dihydroxy-1-naphthaldehyde, 2-phosphate-naphthalene 6-bisphosphonate, 2-naphthol 6-bisphosphonate, 1-phosphate-benzene 4-bisphosphonate, phosphoglycolohydroxamic acid, koningic acid, arsenate, iodoacetate, 3BrPA, DC-5163, Saframycin A, methylglyoxal, NG52, salicylates, MJE3, PGMI-004A, N-Xanthone Benzenesulfonamides, anthraquinone, HKB99, fluoride, SF2312, mefloquine, phosphonoacetohydroxamate. Shikonin, alkannin, the PKM peptide inhibitors TLN-232 and CAP-232, oxamic acid, oxamate, NHI-1, FX11, Quinoline 3-sulfonamides, Trastuzumab, Cetuximab, Bevacizumab, salicylate and its derivatives, 3-phenylpropionate (3PP), 3-(2-methylphenyl)-propionate (2M3PP), or

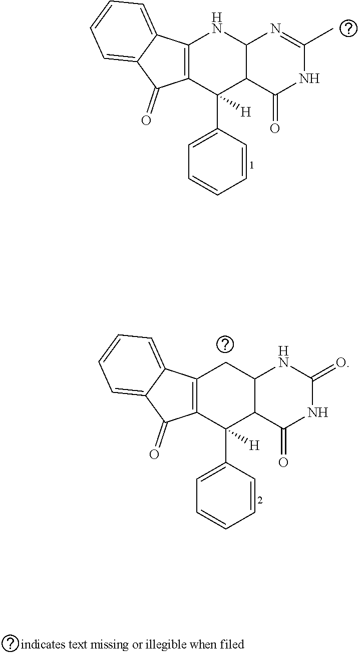 [SMILES: CC1=NC2NC3=C(C(=O)c4ccccc43)[C@H](c3ccccc3)C2C(=O)N1 ; O=C1NC(=O)C2C(CC3=C(C(=O)c4ccccc43)[C@@H]2c2ccccc2)N1]

15. The method according to claim 1, wherein the mitochondrial DNA disorder is associated with a mitochondrial DNA mutation, and wherein the mitochondrial DNA mutation is selected from the group consisting of m.3243A>G, m.8344A>G, m.8993T>G, m.8993T>C, m.583G>A, m.616T>C, m.1494C>T, m.1555A>G, m.1606G>A, m.1630A>G, m.1644G>A, m.3243A>T, m.3256C>T, m.3258T>C, m.3260A>G, m.3271T>C, m.3273delT, m.3280A>G, m.3291T>C, m.3302A>G, m.3303C>T, m.3376G>A, m.3460G>A, m.3635G>A, m.3697G>A, m.3700G>A, m.3733G>A, m.3890G>A, m.3902_3908 ACCTTGCinv, m.4171C>A, m.4298G>A, m.4300A>G, m.4308G>A, m.4332G>A, m.4450G>A, m.5521G>A, m.5537_5538insT, m.5650G>A, m.5690A>G, m.5703G>A, m.5728T>C, m.7445A>G, m.7445A>G, m.7471_7472insC, m.7497G>A, m.7510T>C, m.7511T>C, m.8306T>C, m.8313G>A, m.8340G>A, m.8356T>C, m.8363G>A, m.8528T>C, m.8851T>C, m.8969G>A, m.9035T>C, m.9155A>G, m.9176T>C, m.9176T>G, m.9185T>C, m.9205_9206delTA, m.10010T>C, m.10158T>C, m.10191T>C, m.10197G>A, m.10663T>C, m.11777C>A, m.11778G>A, m.12147G>A, m.12201T>C, m.12258C>A, m.12276G>A, m.12294G>A, m.12315G>A, m.12316G>A, m.12706T>C, m.13042G>A, m.13051G>A, m.13094T>C, m.13379A>C, m.13513G>A, m.13514A>G, m.14459G>A, m.14482C>A, m.14482C>G, m.14484T>C, m.14487T>C, m.14495A>G, m.14568C>T, m.14674T>C, m.14709T>C, m.14710G>A, m.14849T>C, m.15579A>G and m.15990C>T and nt.8467_13446del4977.

16. The method according to claim 1, wherein the mitochondrial DNA disorder is associated with the mitochondrial DNA mutation m.3243A>G, m.8344A>G, m.8993T>G, m.8993T>C or nt.8467_13446del4977.

17. The method according to claim 16, wherein the mitochondrial DNA disorder is associated with the mitochondrial DNA mutation m.3243A>G.

18. The method according to claim 16, wherein the mitochondrial DNA disorder is associated with the mitochondrial DNA mutation nt.8467_13446del4977.

19. The method according to claim 1, wherein the mitochondrial DNA disorder is Maternally Inherited Diabetes and Deafness (MIDD), Mitochondrial Myopathy (MM), Chronic Progressive External Ophthalmoplegia (CPEO), Maternal Inherited Leigh Syndrome (MILS), Mitochondrial Encephalomyopathy Lactic Acidosis and Stroke-Like Episodes (MELAS), Pearson Syndrome (PS), Kearns-Sayre Syndrome (KSS), Myoclonic Epilepsy with Ragged-Red Fibers (MERRF), Neurogenic weakness with Ataxia and Retinitis Pigmentosa (NARP), Mitochondrial NeuroGastroIntestinal Encephalopathy-like (MNGIE-like), Sensory Neural Hearing Loss (SNHL), Sudden Infant Death Syndrome (SIDS), or Focal Segmental Glomerulosclerosis (FSGS).

20. The method according claim 19, wherein the mitochondrial DNA disorder is Mitochondrial Myopathy (MM), Chronic Progressive External Ophthalmoplegia (CPEO), Pearson Syndrome (PS), or Kearns-Sayre Syndrome (KSS).

21. The method according to claim 1, wherein the compound is:
a) administered at a dose of about 2 to about 500 mg/kg body weight, and preferably at a dose of about 20 to about 100 mg/kg body weight;
b) administered at a total dose of about 20 to about 60 mg/kg body weight;
c) administered at a total dose of about 200 to about 1000 international units/m²;
d) administered once daily, twice daily, three times daily or four times daily;
e) administered intermittently every other day, three times per week, twice per week, once per week, once every two weeks, two days every two weeks, three days every two weeks, once every three weeks or once every four weeks;
f) administered orally, intramuscular or intravenously to the patient; or
g) administered orally to the patient.

22-27. (canceled)

28. The method according to claim 27, wherein the compound is administered orally to the patient in the form of a hard or soft capsule, pill, cachet, lozenge, tablet, liquid, powder or granule.

29. The method according to claim 1, wherein the compound is administered to the patient in combination with mannose.

30. The method according to claim 1, wherein the compound is administered to the patient in combination with metformin.

31. (canceled)

32. A method of reducing the mtDNA heteroplasmy in cells of a patient, comprising administering a therapeutically effective amount of a compound to a patient in need thereof, wherein the compound is a glycolysis inhibitor, an inhibitor of glutamine consumption, L-asparaginase, or pegaspargase, thereby reducing mtDNA heteroplasmy in the patient.

33-34. (canceled)

35. A method of reducing the mtDNA heteroplasmy in a cell comprising administering an effective amount of a compound to the cell, wherein the compound is glycolysis inhibitor, an inhibitor of glutamine consumption, L-asparaginase, or pegaspargase, thereby reducing mtDNA heteroplasmy in cell.
